(19) United States
(12) Patent Application Publication
(10) Pub. No.: US 2018/0296070 A1
(43) Pub. Date: Oct. 18, 2018

(54) METHOD FOR OBSERVING THE BRANCH PORTION OF THE HOLE AND METHOD FOR OPERATING THE ENDOSCOPE SYSTEM

(71) Applicant: OLYMPUS CORPORATION, Tokyo (JP)

(72) Inventor: Hiroyuki KOJO, Hachioji-shi (JP)

(73) Assignee: OLYMPUS CORPORATION, Tokyo (JP)

(21) Appl. No.: 15/488,677

(22) Filed: Apr. 17, 2017

(51) Int. Cl. A61B 1/00

(52) U.S. Cl. CPC A61B 1/00071; A61B 1/00009; A61B 1/00114; A61B 1/00064; A61B 1/00002

(57) ABSTRACT

A method for observing the branch portion of the hole include: a step of pushing an obstacle in the hole aside with an elbow portion; a step of rotating a guide member around an axial direction so that a distal portion faces the branch portion extending laterally to an extending direction of the hole; a step of rotating an image to match an up-and-down direction of the image with an up-and-down of a vertical direction, at about the same timing as a timing to rotate the guide member around the axial direction; and a step of projecting an endoscope from the distal portion and inserting into the branch portion while confirming the image.

### 1. FIELD OF THE INVENTION

This invention relates to a method for observing a branch portion of a hole which enables visual recognition of the inside of the hole of an observation target.

### 2. DESCRIPTION OF THE RELATED ART

For example, in US2008/0097154A1, there is disclosed a device which is useful for treatments of diseases of paranasal sinuses. As represented by this device, a surgical procedure to be performed under an endoscope broadly spreads in the treatment of chronic sinusitis.

### BRIEF SUMMARY OF THE INVENTION

A method for observing a branch portion of a hole which uses an endoscope system comprising a guide member having an elbow portion and a distal portion extending laterally from the elbow portion, an endoscope whose orientation is adjustable by the guide member, a controller which processes a signal acquired from the endoscope to generate an image, and a display which displays the image generated by the controller, the method for observing the branch portion of the hole comprising: a step of pushing an obstacle in the hole aside with the elbow portion; a step of rotating the guide member around an axial direction so that the distal portion faces the branch portion extending laterally to an extending direction of the hole; a step of rotating the image to match an up-and-down direction of the image with an up-and-down of a vertical direction, at about the same timing as a timing to rotate the guide member around the axial direction; and a step of projecting the endoscope from the distal portion and inserting into the branch portion while confirming the image.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing an entire configuration of an endoscope system according to a first embodiment;

FIG. 2 is a side view showing, in a perspective manner, the vicinity of a distal constituting portion of an endoscope insertion section of the endoscope system shown in FIG. 1;

FIG. 3 is a sectional view showing the endoscope insertion section in a guide pipe of the endoscope system shown in FIG. 1 which is cut along a plane in an axial direction L;

FIG. 4 is a sectional view schematically showing a state where the endoscope insertion section shown in FIG. 3 is inserted in an opening of a branch portion (a maxillary sinus);

FIG. 5 is a flowchart showing a procedure of a method for observing the branch portion of a hole according to the first embodiment;

FIG. 6 is a schematic view schematically showing the hole (a nasal cavity) of a human head part, the branch portion (the maxillary sinus) branching from the hole, an obstacle (middle nasal concha), and a second obstacle (an uncinate process);

FIG. 7 is a perspective view showing a state just before inserting the guide pipe into a space between the obstacle (the middle nasal concha) that is present in the hole (the nasal cavity) and a wall area defining the periphery of the hole in the endoscope system shown in FIG. 1;

FIG. 8 is a sectional view seen from a distal direction of the guide pipe, concerning positional relations of the obstacle, the guide pipe and the wall area defining the periphery of the hole in a state shown in FIG. 7;

FIG. 9 is a perspective view showing, with a two-dot chain line, the guide pipe of a state prior to rotation, and showing, with a solid line (a partially broken line), a state where the guide pipe is rotated as much as 60° to 90° to ride over the second obstacle (the uncinate process) in a state where the distal portion of the guide pipe is located in the vicinity of an opening of the branch portion (the maxillary sinus);

FIG. 10 is a schematic view showing, from a distal side, the guide pipe rotated as much as 60° to 90° from its state shown in FIG. 9, and showing a relation between an up-and-down direction of an image obtainable from an endoscope and an up-and-down of a vertical direction;

FIG. 11 is a schematic view showing the guide pipe that is present at a position similar to that of FIG. 9, and showing a relation obtained by executing step S14 of FIG. 5 to match the up-and-down direction of the image with the up-and-down of the vertical direction;

FIG. 12 is a schematic view showing relations of each direction of the image (the image prior to the execution of the step S14) obtained from the endoscope shown in FIG. 10, an up-and-down of the vertical direction, and an anterior posterior direction;

FIG. 13 is a schematic view showing relations of each direction of the image (the image after the step S14 is executed) obtained from the endoscope shown in FIG. 11, the up-and-down of the vertical direction, and the anterior posterior direction;

FIG. 14 is a schematic view showing a state where the endoscope and the guide pipe are inserted into a hole (left nasal cavity) and the branch portion (a paranasal sinus and left maxillary sinus) extending laterally from a hole extending direction of a medical examinee in a recumbent posture in a first example of the method for observing the branch portion of the hole (a method for operating the endoscope system);

FIG. 15 is a schematic view showing the left nasal cavity shown in FIG. 14 and further showing the inside of the left maxillary sinus in a perspective manner;

FIG. 16 is a schematic view showing an image (an image of a state prior to rotation at an angle) obtained from the endoscope in the state shown in FIG. 14;

FIG. 17 is a schematic view showing an image after the image shown in FIG. 16 is rotated at the angle;

FIG. 18 is a schematic view showing a state where the endoscope and the guide pipe are inserted into a hole (right nasal cavity) and a branch portion (a paranasal sinus and right maxillary sinus) of a medical examinee in a recumbent posture in a second example of the method for observing the branch portion of the hole (the method for operating the endoscope system);

FIG. 19 is a schematic view showing the right nasal cavity shown in FIG. 18, and further showing the inside of the right maxillary sinus in a perspective manner;

FIG. 20 is a schematic view showing an image (an image of a state prior to rotation at an angle) obtained from the endoscope in the state shown in FIG. 18;

FIG. 21 is a schematic view showing an image after the image shown in FIG. 20 is rotated at the angle;

FIG. 22 is a schematic view showing a state where the endoscope and the guide pipe are inserted into a hole (left nasal cavity) and a branch portion (a paranasal sinus and left maxillary sinus) of a medical examinee in a seated posture in a third example of the method for observing the branch portion of the hole (the method for operating the endoscope system);

FIG. 23 is a schematic view showing the left nasal cavity shown in FIG. 22, and further showing the inside of the left maxillary sinus in a perspective manner;

FIG. 24 is a schematic view showing an image (an image of a state prior to rotation at an angle) obtained from the endoscope in the state shown in FIG. 22;

FIG. 25 is a schematic view showing an image after the image shown in FIG. 24 is rotated at the angle;

FIG. 26 is a sectional view schematically showing a guide pipe and an endoscope insertion section of an endoscope system of a second embodiment;

FIG. 27 is a sectional view schematically showing a state where an operation (a bending operation) of noticeably bending the endoscope insertion section toward a back side is performed in the endoscope system shown in FIG. 26;

FIG. 28 is a sectional view schematically showing a state where the endoscope insertion section is projected from the guide pipe and inserted in an opening of a branch portion (a maxillary sinus), in the endoscope insertion section and the guide pipe shown in FIG. 27; and

FIG. 29 is a flowchart showing a procedure of a method for observing the branch portion of a hole according to the second embodiment.

### DETAILED DESCRIPTION OF THE INVENTION

### First Embodiment

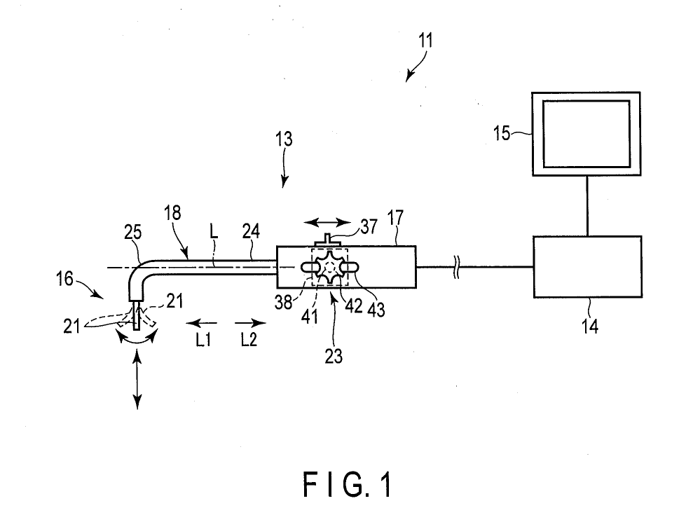

Hereinafter, there will be described a first embodiment of an endoscope system and a method for observing a branch portion of a hole which uses the endoscope system, with reference to FIG. 1 to FIG. 13. Hereinafter, it will be described that the endoscope system and the method for observing the branch portion of the hole which uses the endoscope system are applied to so-called medical purposes.

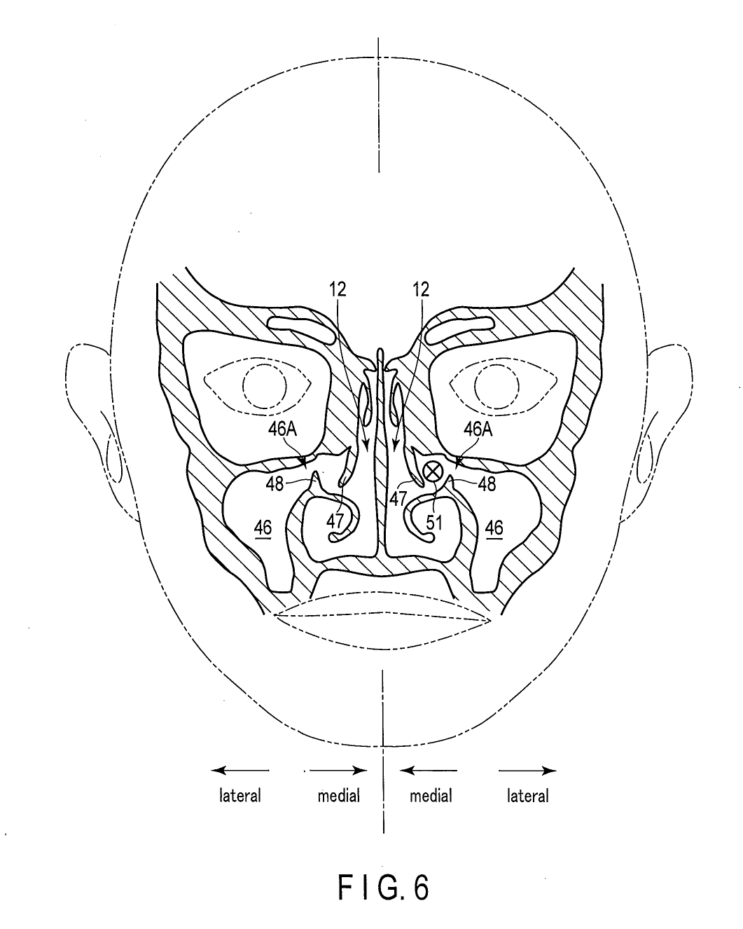

As shown in FIG. 1, an endoscope system 11 includes an insertion device 13 to be inserted into a hole 12 (e.g., a nasal cavity or the like as shown in FIG. 6) of a subject and used, a controller 14 electrically connected to the insertion device 13, and a display 15 connected to the controller 14. The insertion device 13 is provided separately from the display 15 and the controller 14. The display 15 is constituted of a usual liquid crystal monitor and is capable of displaying an image acquired from an endoscope 16.

As shown in FIG. 1 to FIG. 3, the insertion device 13 includes a holding section 17 forming a device handle portion and constituting an outer shell, a tubular guide pipe 18 protruding from the holding section 17, an endoscope insertion section 21 passed through the guide pipe 18 and the holding section 17, an endoscope imaging section 22 (an imaging section) provided inside the holding section 17, and a curve operation section 23 provided in the holding section 17. The holding section 17 forms a cylindrical shape and constitutes a portion (a housing) to be held with a hand of a user (a surgeon).

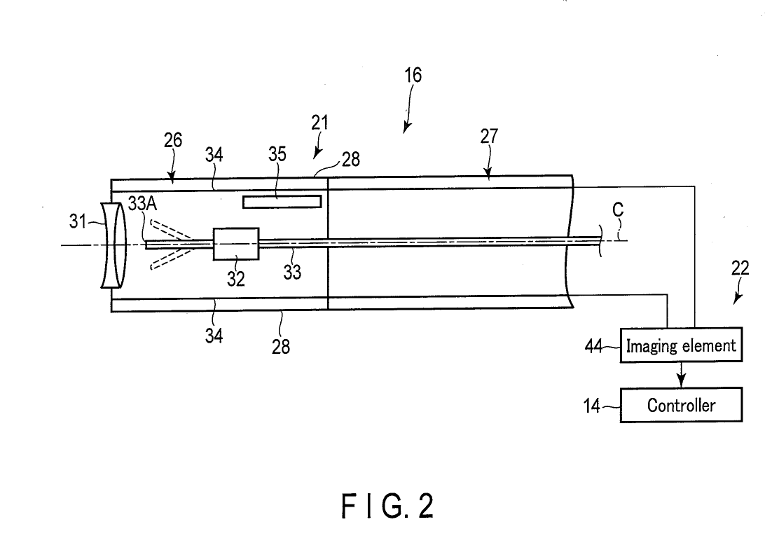

In the present embodiment, as shown in FIG. 2, the endoscope 16 is separated into two sections, i.e., the endoscope insertion section 21 and the endoscope imaging section 22, but may integrally be constituted of these sections. As shown in FIG. 1, it is assumed that an axial direction (a central axis direction) of an after-mentioned main body portion 24 of the guide pipe 18 is L, and in the axial direction L, an after-mentioned direction from the holding section 17 toward an elbow portion 25 is L1 and a direction from the elbow portion 25 toward the holding section 17 is L2, whereby description will be made.

In the present embodiment, the endoscope 16 is constituted of a so-called scanning type endoscope. The endoscope 16 (the endoscope insertion section 21) is flexibly constituted. Consequently, the endoscope insertion section 21 is passed through the guide pipe 18, and is therefore bendable in accordance with a shape of the guide pipe 18. An orientation of the endoscope insertion section 21 is adjustable by the guide pipe 18. As shown in FIG. 2, the endoscope insertion section 21 includes a central axis C. As shown in FIG. 1 and FIG. 4, the endoscope insertion section 21 moves along a central axis C direction to be projected from the guide pipe 18.

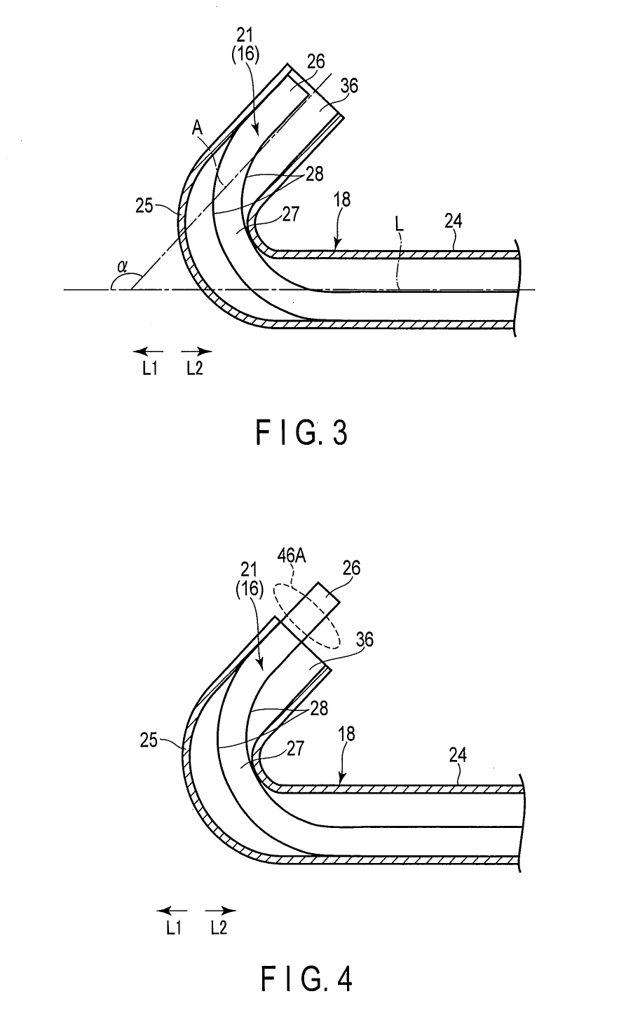

As shown in FIG. 2 and FIG. 3, the endoscope insertion section 21 includes a distal constituting portion 26 located on a distal side of the central axis C direction, a flexible tube 27 provided on a proximal side of the central axis C direction with respect to the distal constituting portion 26, a pair of wires (pull wires) provided on the right and the left across a distal end of a sheath 28 and the holding section 17, the tubular sheath 28 which covers the distal constituting portion 26, the flexible tube 27 and the wires, an illumination window 31, a rotating unit 32, an illuminating fiber 33, light receiving fibers 34, and a sensor 35 received in the distal constituting portion 26. In the present embodiment, the sheath 28 is constituted to be bendable in a right-left direction (or an up-and-down direction) as shown in FIG. 1, together with the endoscope insertion section 21 (the flexible tube 27) held inside the sheath. The sensor 35 is constituted of a commercially available gravity sensor (acceleration sensor). The sensor 35 is capable of detecting an angle (tilt) to be formed by a line segment A (i.e., the distal constituting portion 26) connecting a distal portion 36 of the guide pipe 18 and the elbow portion 25 to a vertical direction, when the endoscope insertion section 21 is received in the guide pipe 18 and is not bent.

As shown in FIG. 1, the holding section 17 may further be provided with an advancing/retreating mechanism 37 which advances and retreats the endoscope insertion section 21 in the axial direction L through the guide pipe 18. The advancing/retreating mechanism 37 is constituted of, for example, a knob portion capable of advancing and retreating a support unit 38. That is, the user who is the surgeon can utilize the advancing/retreating mechanism 37 or the like to change a position of the endoscope insertion section 21, or to change a bend angle of the endoscope insertion section 21 as described later, in a state where the endoscope insertion section 21 is inserted in the hole 12 (the nasal cavity or the like) of a medical examinee in diagnosis, so that a desirable image of the inside of the hole 12 is obtainable.

The curve operation section 23 includes the support unit 38 received in the holding section 17 to be slidable in the axial direction L of the guide pipe 18, a shaft portion 41 supported to be rotatable to the support unit 38, a dial 42 (a knob or a rotating knob) fixed to one end portion of the shaft portion 41, and an unshown sprocket fixed to the other end portion of the shaft portion 41 inside a case of the support unit 38. The shaft portion 41 protrudes from a long hole 43 formed in the holding section 17 to the outside of the holding section 17. An end portion of the above wire is connected to a chain which can mesh with the sprocket. With the rotation of the dial 42, one of the above pair of wires is pulled, and the other wire loosens, whereby the distal end of the sheath 28 is pulled and the sheath 28 bends to the right or the left in FIG. 1. When the sheath 28 bends, the endoscope insertion section 21 (the flexible tube 27) which is present in the sheath also bends. A bending direction of the sheath 28 is merely one example, and the sheath 28 may bend to a distal side and a proximal side of a paper surface in FIG. 1, or needless to say, the wire may be bendable in four directions of the upward, downward, right and left directions. By pulling the wire (a linear member), the bend angle of the endoscope 16 (the endoscope insertion section 21) is adjustable.

As shown in FIG. 2, the illuminating fiber 33 is optically connected to a light source provided adjacently to the controller 14. The light receiving fibers 34 are optically connected to an imaging element 44. Distal ends of the light receiving fibers 34 are exposed to the outside in the vicinity of the distal constituting portion 26. Consequently, the endoscope 16 can acquire the image via the light receiving fibers 34 in the distal constituting portion 26.

As shown in FIG. 2, the endoscope imaging section 22 includes the imaging element 44 constituted of a CCD, a CMOS, and others. The endoscope imaging section 22 can acquire the image obtained with the distal constituting portion 26 of the endoscope insertion section 21. More specifically, the imaging element 44 converts light from the light receiving fibers 34 into an electric signal to send the signal to the controller.

The rotating unit 32 is electrically connected to the controller 14. The rotating unit 32 is constituted of a motor and others, and is, for example, spirally swung under control of the controller 14. Consequently, a distal end 33A of the illuminating fiber 33 is spirally swung in accordance with an operation of the rotating unit 32. Therefore, the surface of the subject is spirally scanned with illumination light from the illuminating fiber 33 through the distal end of the illuminating fiber 33 and the illumination window 31. The light receiving fibers 34 receive light returned from the subject to guide the light to the imaging element 44. The imaging element 44 converts the light received by the light receiving fibers 34 into the electric signal to send the signal to the controller 14. The controller 14 converts the electric signal into the image, and suitably performs image processing to display the image in the display 15.

As shown in FIG. 3, the guide pipe 18 substantially forms an “L”-shape or “J”-shape as a whole, and forms a tubular shape (a cylindrical shape) halfway bent in the form of an elbow. The guide pipe 18 includes the main body portion 24 having one end portion attached to the holding section 17, the elbow portion 25 provided in the other end portion of the main body portion 24, and the distal portion 36 protruding from the elbow portion 25 in a direction away from the main body portion 24 (i.e., in a lateral direction). An angle α to be formed by the line segment A connecting the distal portion 36 and the elbow portion 25 to the direction L1 which comes closer to the elbow portion 25 in the axial direction L (the central axis direction) of the main body portion 24 is, for example, a right angle or an obtuse angle, and is more specifically 90°≤α≤120°. Therefore, in the present embodiment, the endoscope insertion section 21 (the endoscope 16) can be disposed in a direction that forms the right angle or the obtuse angle to the direction L1 which comes closer to the elbow portion 25, depending on the shape of the guide pipe 18.

The endoscope insertion section 21 can be passed through the guide pipe 18. Along an inner wall of the guide pipe 18, the endoscope insertion section 21 which moves to advance and retreat along the central axis C can be guided. The guide pipe 18 is one example of a guide member. It is preferable that the guide pipe 18 is provided fixedly to, for example, the holding section 17, but may be rotatable around the axial direction L to the holding section 17. In this case, the holding section 17 may be provided with a rotating knob to rotate the guide pipe 18 around the axial direction L.

The controller 14 shown in FIG. 1 is constituted of, for example, a usual computer and software installed in this computer to execute various types of control of the insertion device. The controller 14 can execute control to respective sections of the insertion device 13, for example, as follows. The controller 14 can control the rotating unit 32 that swings the illuminating fiber 33 to adjust the number of rotations and the like of the rotating unit. The controller 14 can control the light source to adjust a quantity of the light to be supplied to the illuminating fiber 33. The controller 14 processes the electric signal corresponding to the image acquired with the imaging element of the insertion device 13 to form the image, and can display an image 45 in the display 15. Furthermore, the controller 14 can rotate the image 45 to match an up-and-down direction of the image 45 with an up-and-down of the vertical direction when angular information (the tilt) of the distal constituting portion 26 which is obtained from the sensor 35 is in excess of a predetermined threshold value. The controller 14 stores the predetermined threshold value required for this control. The predetermined threshold value can take an appropriate value in a range of, for example, 40° to 50°, and is set to, for example, 45° or the like in the present embodiment.

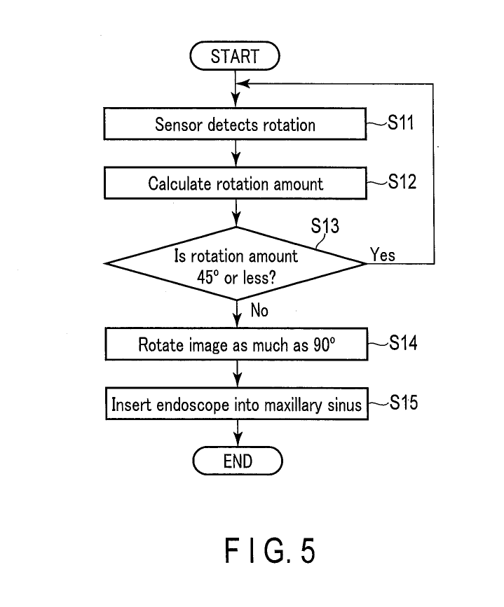

Next, there will be described the method for observing the branch portion of the hole which uses the endoscope system 11 of the present embodiment, with reference to FIG. 5 to FIG. 11. A flowchart of FIG. 5 shows respective steps of the present observing method, and the description will be made with reference to this flowchart. It is to be noted that here, there is described an example where the method for observing the branch portion of the hole is applied to observation of a paranasal sinus (a maxillary sinus) extending laterally from the nasal cavity of the medical examinee. Furthermore, it is assumed that the medical examinee who sits up on a seat plane receives the observation by the user who is the surgeon.

As shown in FIG. 6, the maxillary sinus in the paranasal sinus constitutes a branch portion 46 extending laterally from the nasal cavity (the hole 12), the nasal cavity (the hole 12) being substantially positioned in a medial area of a face and extending in an anterior posterior direction. An area between the nasal cavity and the maxillary sinus has middle nasal concha that is an organ dividing off this area, and an uncinate process that is an organ dividing off the area. The uncinate process is positioned further laterally with respect to the middle nasal concha. When observing the inside of the maxillary sinus (the branch portion 46), the user can recognize the middle nasal concha and the uncinate process as obstacles in reaching the maxillary sinus. Consequently, in the present description, it is assumed that the middle nasal concha is conveniently an obstacle 47 and the uncinate process is a second obstacle 48 provided before the branch portion 46 in the hole 12, whereby the description will be made. It is also assumed that the nasal cavities are the holes 12 and that the maxillary sinuses are the branch portions 46, whereby the description will be made. It is to be noted that an opening 46A of each branch portion 46 (the maxillary sinus) is present in the vicinity of the second obstacle 48 on a lateral side with respect to the second obstacle 48.

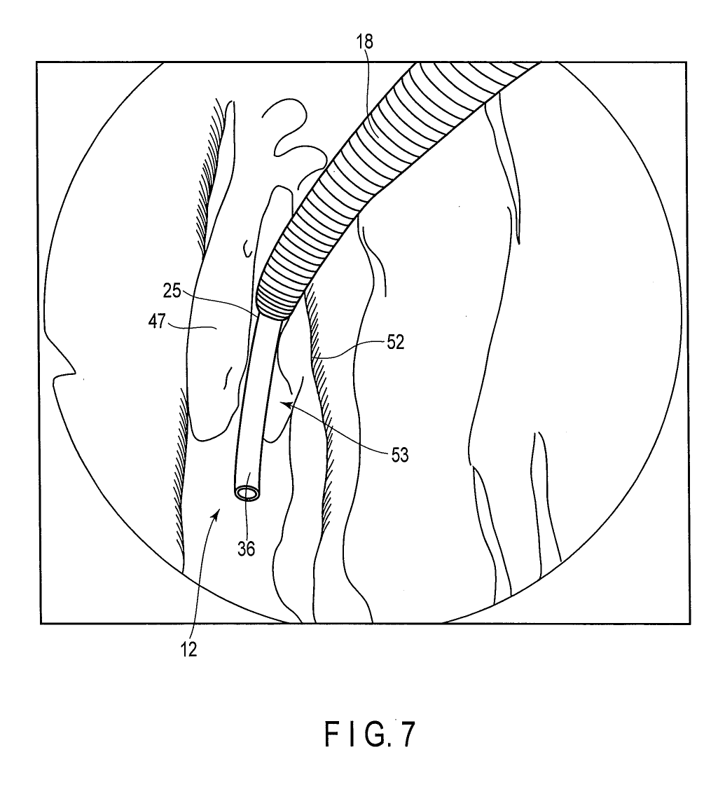

The user who is the surgeon can insert the guide pipe 18 into the hole 12 from an external nostril of the medical examinee (the subject) in diagnosis. FIG. 6 shows a position of the inserted guide pipe 18 with an arrowhead 51. FIG. 6 shows a state where the guide pipe 18 is inserted in left hole 12 (left nasal cavity) of the medical examinee. As shown in FIG. 7, in the hole 12, the obstacle 47 (the middle nasal concha) is present before the branch portion 46 (the maxillary sinus) is reached. FIG. 7 shows an image obtained by photographing the guide pipe 18 and the obstacle 47 (the middle nasal concha) with an unshown second endoscope inserted from the external nostril. In this method for observing the branch portion of the hole, it is possible to observe the branch portion 46 without using such a second endoscope, but the observation of the branch portion 46 may be assisted by looking down upon the endoscope insertion section 21 and the guide pipe 18 with the second endoscope that is capable of photographing such an image as shown in FIG. 7.

As shown in FIG. 7, the obstacle 47 (the middle nasal concha) is located to hang down from a wall area 52 (a part of a human body) defining a periphery of the hole 12 (the nasal cavity) to the downside of the vertical direction. Here, the user can insert the guide pipe 18 into a space 53 between the wall area 52 defining the periphery of the hole 12 and the obstacle 47. Thus, the distal portion of the guide pipe 18, directed to downside in the vertical direction can be advanced toward a head rear (posterior) side of the medical examinee while pushing the obstacle 47 aside with the elbow portion 25.

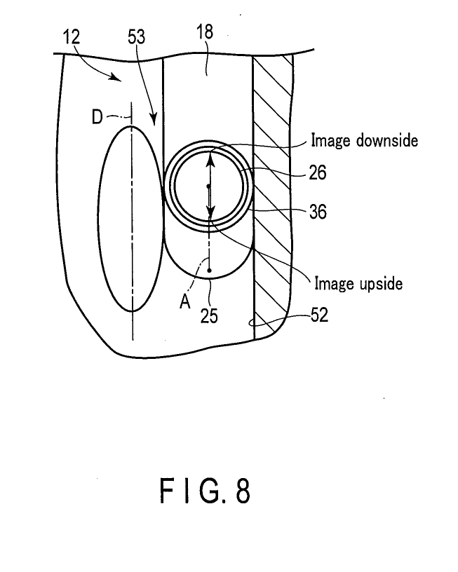

FIG. 8 is a sectional view of the guide pipe 18, the obstacle 47, and the wall area 52 defining the periphery of the hole 12 which are seen from the downside of the vertical direction. The user may advance the distal portion 36 of the guide pipe 18 toward the head posterior side of the medical examinee while directing the distal portion 36 of the guide pipe 18 toward the upside of the vertical direction to push the obstacle aside with the distal portion 36. It is to be noted that when the guide pipe 18 is advanced and retreated in a state where the distal portion 36 of the guide pipe 18 is directed to the downside of the vertical direction (or the upside of the vertical direction), there is obtained a situation closely similar to a state where a person walks looking downward (or upward), and hence user's intuition is matched. That is, the upside of a viewing field (the image) corresponds to the head posterior side of the medical examinee (an advancing direction side), the downside of the viewing field (the image) corresponds to a head anterior side of the medical examinee (a side reverse to an advancing direction), the right and left correspond to those as they are, and hence the closely similar situation is obtainable. At this time, as shown in FIG. 8, the elbow portion 25 and the distal portion 36, i.e., the line segment A connecting the distal portion 36 and the elbow portion 25 is substantially parallel with a plane D along which the obstacle 47 extends.

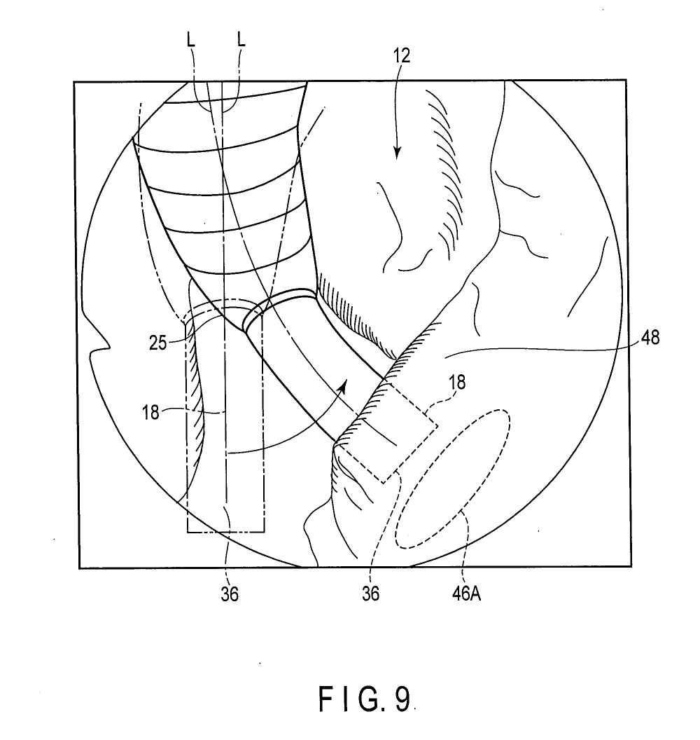

When bringing the distal portion 36 of the guide pipe 18 closer to the opening 46A of the branch portion 46, the user twists the holding section 17 as much as about 60° to 90° around the axial direction L while pushing the obstacle 47 aside with the elbow portion 25 as shown in FIG. 9, whereby the guide pipe 18 can be rotated around the axial direction L to direct the distal portion 36 laterally from the downside of the vertical direction. Consequently, the distal portion 36 is laterally directed to ride over the second obstacle 48 and can be opposed to the opening 46A of the branch portion 46. When advancing the distal portion 36 of the guide pipe 18 directed to the upside in the vertical direction, the user twists the holding section 17 as much as about 60° to 90° around the axial direction L, whereby the distal portion 36 can be opposed to the opening 46A of the branch portion 46 while the distal portion 36 is laterally directed to ride over the second obstacle 48. Thus, the distal portion 36 can hold the second obstacle 48 in a state where the endoscope insertion section 21 (the endoscope 16) is insertable in the branch portion 46.

In circumstances where the distal portion 36 is laterally directed, the sensor 35 can detect that the guide pipe 18 (the distal constituting portion 26) is rotated (step S11). The sensor 35 then detects an angle of the rotation (tilt) of the distal portion 36 of the guide pipe 18 to the downside of the vertical direction, to send angular information of the guide pipe 18 to the controller 14. The controller 14 calculates a rotation amount (a rotation angle) of the guide pipe 18 (the distal constituting portion 26) from the downside of the vertical direction on the basis of the angular information from the sensor 35 (step S12). The controller 14 judges whether the above rotation amount of the guide pipe 18 is a threshold value (e.g., 45°) or less, or is in excess of the threshold value (e.g., 45°) (step S13), and the controller rotates the image 45 displayed in the display 15 as much as 90° in a clockwise direction in a case where the above rotation amount of the guide pipe 18 is in excess of the threshold value (e.g., 45°) (step S14).

At this time, in a direction to rotate the image 45, the upside of the image 45 corresponds to the upside of the vertical direction and the downside of the image 45 corresponds to the downside of the vertical direction. Needless to say, the angle to rotate the image 45 at this time may be about 90° (e.g., from 60° to 120° and preferably from 75° to 105°). In a case where the angle to rotate the image 45 is about 90°, the upside of the vertical direction does not exactly match the upside of the image 45, but both positions are approximately matched. Consequently, the user can intuitively recognize the situation of the inside of the hole 12. Furthermore, the rotation of the image 45 is performed in image processing by the controller 14, but the image 45 obtained by mechanically rotating the imaging element 44 may be rotated. A timing to rotate the image 45 is about the same as a timing to rotate the guide pipe 18 around the axial direction L. About the same timing mentioned here is a timing of 1/10 to 1/1000 seconds after the sensor 35 detects the rotation of the guide pipe 18 and the controller 14 judges that the above rotation amount of the guide pipe 18 is in excess of the threshold value (i.e., substantially simultaneously with the rotation of the guide pipe 18). Alternatively, about the same timing mentioned here may be a timing of several seconds after the sensor 35 detects the rotation of the guide pipe 18 and the controller 14 judges that the rotation amount of the guide pipe 18 is in excess of the threshold value. Furthermore, the controller 14 may predict the movement of the guide pipe 18 that is to be rotated, from information (e.g., acceleration of the guide pipe 18) obtained from the sensor 35 or the like. In this case, about the same timing may be a timing to actually rotate the image 45 beforehand prior to completion of the rotation of the guide pipe 18 around the axial direction L.

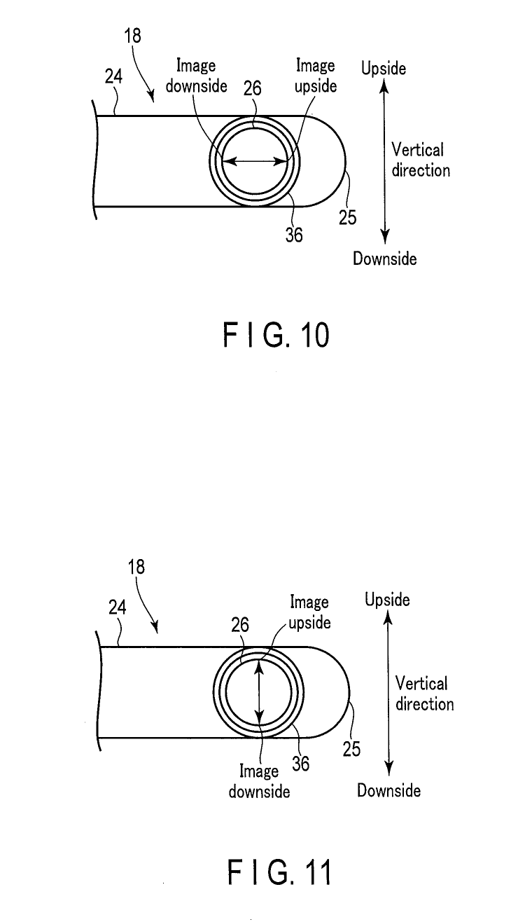

FIG. 10 and FIG. 11 are views schematically showing the guide pipe 18 and the endoscope insertion section 21 from a direction opposed to the distal portion 36 of the guide pipe 18, and schematically showing a situation to match the up-and-down direction of the image 45 with the up-and-down of the vertical direction in the step S14. That is, in FIG. 10, the up-and-down direction of the image 45 does not match the up-and-down of the vertical direction, but when the control of the step S14 is performed, the up-and-down direction of the image 45 matches the up-and-down of the vertical direction as shown in FIG. 11.

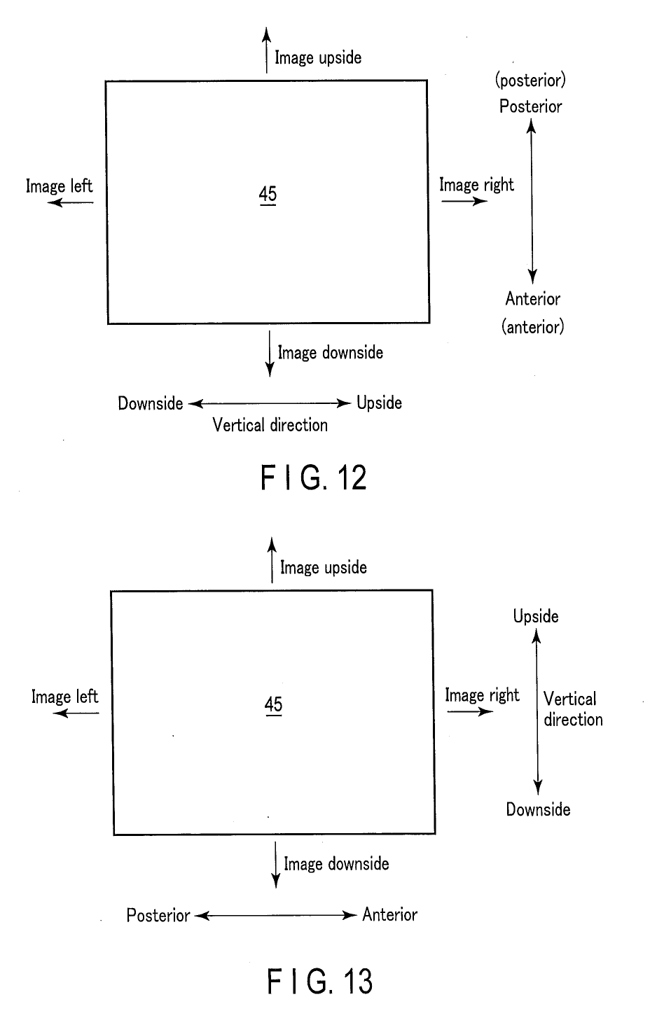

This situation will be described in more detail with reference to FIG. 12 and FIG. 13. As shown in FIG. 12, in the situation prior to the rotation of the image 45, the up-and-down direction of the image 45 extends along the anterior posterior direction, and therefore the image does not match the user's intuition. For example, when performing an operation of pushing and pulling the holding section 17, the image 45 is to be scrolled in the up-and-down direction, and the user cannot intuitively recognize this operation. Similarly, when performing an operation of twisting the holding section 17 around the axial direction L as much as a minor angle to change the angle (tilt) of the distal portion 36 of the guide pipe 18, the image 45 is to be scrolled in the right-left direction, and the user cannot intuitively recognize this operation. This also applies to a case of moving the holding section 17 upward and downward in the vertical direction to move the position of the distal portion 36 of the guide pipe 18 upward and downward in the vertical direction, and the user cannot intuitively recognize this operation.

On the other hand, as shown in FIG. 13, the upside of the image 45 matches the upside of the vertical direction and the downside of the image 45 matches the downside of the vertical direction, after the image 45 is rotated in the step S14. Consequently, for example, when performing the operation of pushing and pulling the holding section 17, the image 45 is to be scrolled in the right-left direction, and the user can intuitively recognize the situation of the inside of the hole 12. Similarly, when performing the operation of twisting the holding section 17 around the axial direction L as much as the minor angle to change the angle (tilt) of the distal portion 36 of the guide pipe 18, the image 45 is also scrolled in the up-and-down direction, and the user can intuitively recognize the situation of the inside of the hole 12. Also when moving the holding section 17 upward and downward in the vertical direction to move the position of the distal portion 36 of the guide pipe 18 upward and downward in the vertical direction, the image 45 is to be scrolled in the up-and-down direction, and the user can intuitively recognize the situation of the inside of the hole 12.

In a case where the above rotation amount of the guide pipe 18 is not more than the threshold value (e.g., 45°), the image 45 to be displayed in the display 15 is not changed, but the sensor 35 continues to monitor presence/absence of the rotation of the guide pipe 18 (the step S11).

There is obtained a situation closely similar to a state where one turns one's neck in a lateral direction (to the right side), after the image 45 is rotated. That is, the up-and-down direction of the image 45 match the up-and-down of the vertical direction. Furthermore, the left side of the image 45 corresponds to a posterior side of a head of the medical examinee (the advancing direction side) and the right side of the image 45 corresponds to the head anterior side of the medical examinee. Consequently, the user can intuitively recognize the situation of the inside of the hole 12. In this state, the user utilizes the image 45 to minutely adjust the tilt of the endoscope insertion section 21 or the position of the distal constituting portion 26 as required. The user projects the endoscope insertion section 21 (the endoscope 16) from the distal portion 36 of the guide pipe 18 and inserts the endoscope insertion section 21 into the opening 46A of the branch portion 46 (the maxillary sinus) as shown in FIG. 4, while confirming the image 45 obtained from the endoscope 16 (step S15). Consequently, the user can observe the situation of the inside of the branch portion 46 (the maxillary sinus). After ending the observation of the inside of the branch portion 46, the user receives the endoscope insertion section 21 in the guide pipe 18 and twists the holding section as much as about 60° to 90° to direct the distal portion 36 of the guide pipe 18 to the downside of the vertical direction (or the upside of the vertical direction). It is preferable that the controller 14 returns the image 45 to its state prior to the rotation to match the user's intuition after the distal portion 36 of the guide pipe 18 is directed to the downside of the vertical direction (or the upside of the vertical direction).

More specifically, the sensor 35 detects the presence/absence of the rotation of the guide pipe 18 (the distal constituting portion 26), and returns the image 45 to its state prior to the rotation in a case where the angle (tilt) of the rotated guide pipe 18 (distal constituting portion 26) is not more than the threshold value (e.g., 45°) from the downside of the vertical direction. Also in the state where the distal portion 36 of the guide pipe 18 is directed to the downside of the vertical direction (or the upside of the vertical direction), the image 45 obtained from the endoscope 16 matches the user's intuition. Consequently, the user can safely remove the guide pipe 18 from the hole 12 (the nasal cavity). The user can perform a procedure similar to the above method also in inserting the guide pipe 18 into the right hole 12 (the right nasal cavity) of the medical examinee to observe the right branch portion 46 (the right maxillary sinus). In this case, a direction in which the image 45 displayed in the display 15 rotates is different from the above direction, and in the step S14, the image rotates as much as 90° (or about 90°) in a counterclockwise direction. Furthermore, it is preferable that the controller 14 stores a program corresponding to a first mode to observe the left branch portion 46 of the medical examinee and a program corresponding to a second mode to observe the right branch portion 46 of the medical examinee. It is preferable that the controller 14 or the holding section 17 is provided with a switch (a push button) which is switchable to the first mode and the second mode.

The angle at which the image 45 rotates in the clockwise or counterclockwise direction is not restricted to the preset angle described above, and as described below in a first example to a fourth example, each appropriate rotation angle may be calculated on the basis of an angle at which the endoscope insertion section 21 (the guide pipe 18 and the distal constituting portion 26) is presently disposed.

### First Example

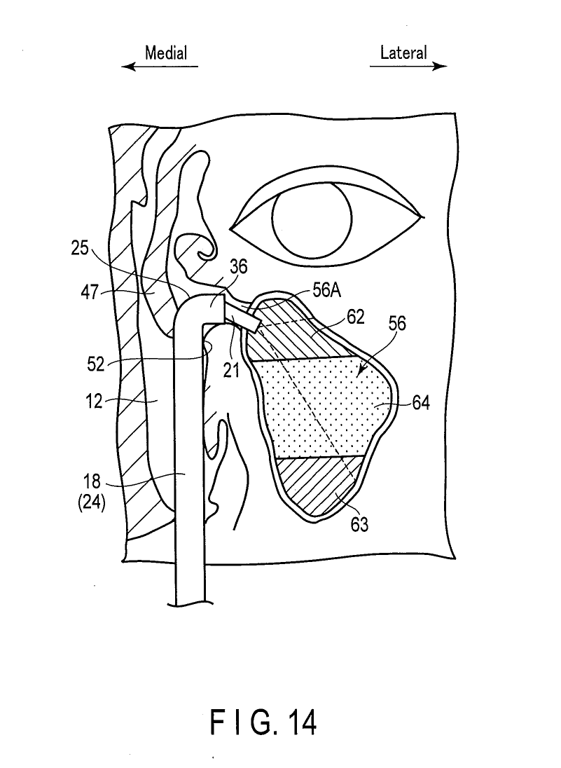

Next, there will be described the first example of the method for observing the branch portion of the hole which uses the endoscope system 11 of the present embodiment (the method for operating the endoscope system), with reference to FIG. 14 to FIG. 17. It is assumed that the medical examinee in the recumbent posture (in a state of lying on a bed) has left maxillary sinus observed by the user who is the surgeon as shown in FIG. 14. Furthermore, the observation is performed in a recumbent posture first mode that is different from the above-mentioned first mode. For the switch to the recumbent posture first mode, the switch (the push button) is usable.

A procedure similar to the above embodiment is executable until the guide pipe 18 is inserted into the space 53 between the wall area 52 defining the periphery of the hole 12 and the obstacle 47. In the first example, the distal portion 36 of the guide pipe 18 (the distal constituting portion 26) is directed to a jaw side in the state shown in FIG. 7. At this time, the controller 14 acquires information on an extending direction (angle) of the distal portion 36 (the distal constituting portion 26) via the sensor 35 to store the information, so that a jaw side (inferior) direction (angle) can be stored. As a trigger to store the jaw side (inferior) direction in the controller 14, a button operation or the like from the user is utilizable. It is preferable that the button is provided in the holding section 17 or the like. For the purpose of storing the jaw side (inferior) direction in the controller 14, the controller 14 may display whether or not “the jaw side direction is to be stored” in the display 15 after the above mode is selected with the switch.

As shown in FIG. 9, the user twists the holding section 17 as much as about 60° to 90° around the axial direction L in a state where the guide pipe 18 is located in the vicinity of a branch portion 56, whereby the guide pipe 18 can be rotated around the axial direction L to direct the distal portion 36 laterally from the jaw side. Thus, the distal portion 36 is laterally directed to ride over the second obstacle 48 and can be opposed to (face) an opening 56A of the branch portion 56. Consequently, the distal portion 36 can hold the second obstacle 48 in a state where the endoscope insertion section 21 (the endoscope 16) is insertable in the branch portion 56.

It is to be noted that the sensor 35 detects a degree of tilt of the vicinity of the distal end of the endoscope 16 (the distal constituting portion 26, the holding section 17 and the guide pipe 18) to the jaw (inferior) side, to always send the information to the controller 14. Consequently, also in circumstances where the distal portion 36 is laterally directed, the sensor 35 sends, to the controller 14, angular information indicating the degree of the tilt of the vicinity of the distal end of the endoscope 16 (the distal constituting portion 26, the holding section 17 and the guide pipe 18) to the jaw side. The controller 14 judges whether the tilt of the distal portion 36 (the distal constituting portion 26) to the jaw side is a threshold value (e.g., 45°) or less, or is in excess of the threshold value (e.g., 45°).

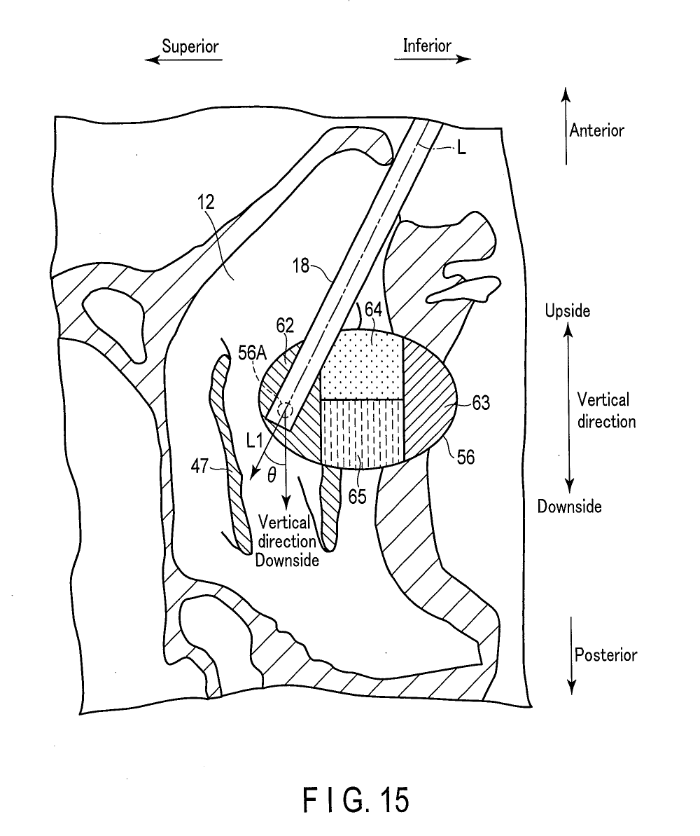

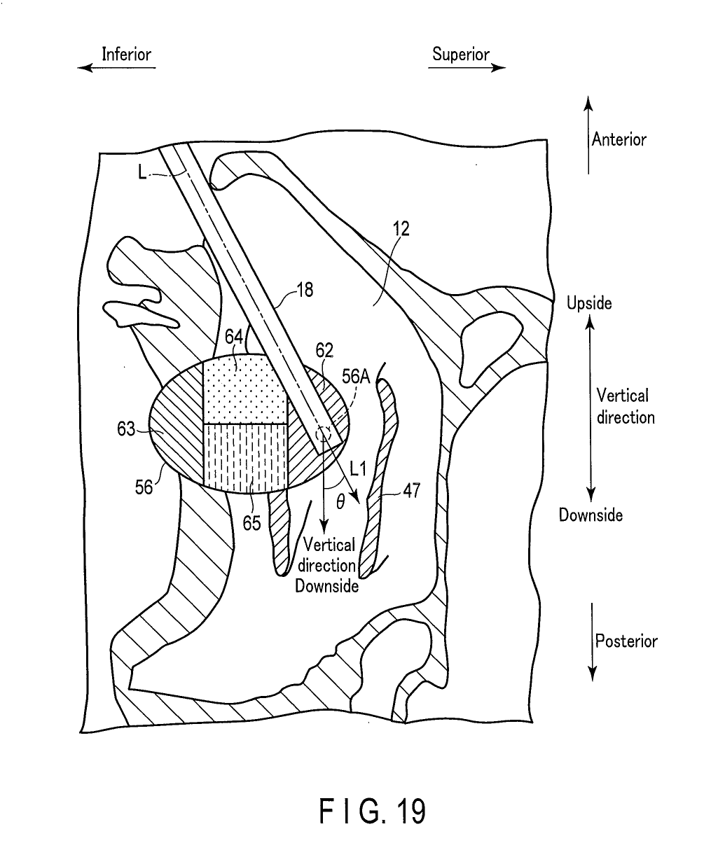

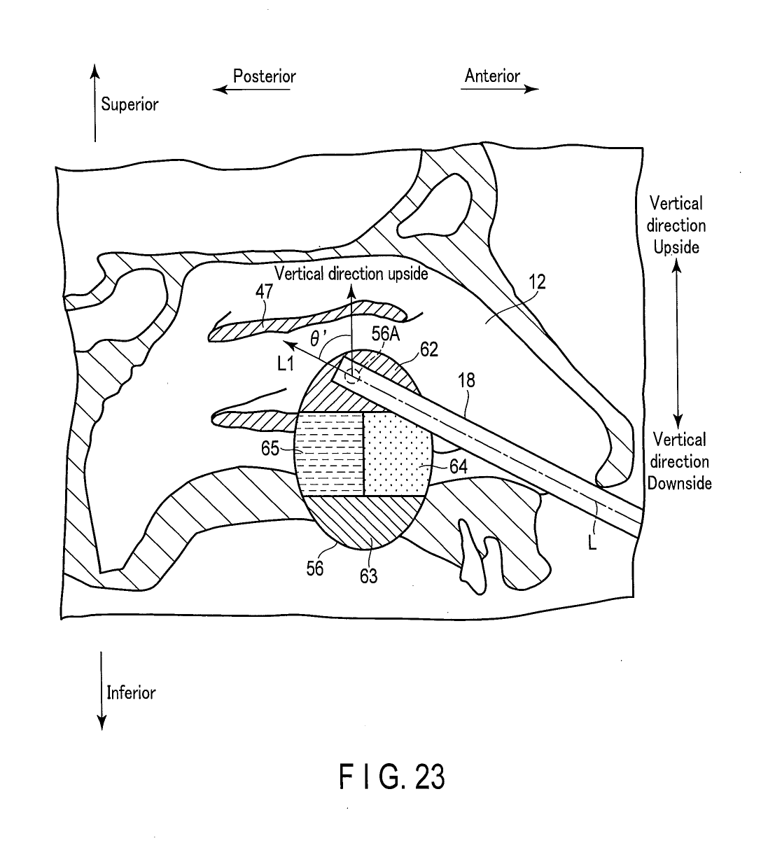

The controller 14 rotates the image 45 displayed in the display 15 when obtaining the judgment result which is in excess of the above threshold value. The controller 14 rotates the image 45 around its central area to match the downside of the vertical direction detected by the sensor 35 with the downside of the image 45 recognized by the user and to match the upside of the vertical direction detected by the sensor 35 with the upside of the image 45 recognized by the user. For example, as shown in FIG. 15, the image 45 is rotated as much as 180−θ° in the counterclockwise direction at this time, in which θ° is an angle to be formed by the direction L1 which comes closer to the elbow portion 25 of the guide pipe 18 and the downside of the vertical direction. In the first to fourth examples, as it is clear from FIG. 15, FIG. 19 and FIG. 23, the line segment A connecting the distal portion 36 of the guide pipe 18 and the elbow portion 25 (i.e., the distal constituting portion 26) is almost superimposed on the axial direction L of the guide pipe 18, and hence needless to say, it is considered that the sensor 35 present in the distal constituting portion 26 can detect the angle (the tilt) to be formed by the axial direction L of the guide pipe 18 and the vertical direction. On the other hand, when the controller 14 obtains the judgment result which is not more than the above-mentioned threshold value, the rotation of the image 45 displayed in the display 15 is not performed.

At this time, a timing to rotate the image 45 is about the same as a timing to rotate the guide pipe 18 around the axial direction L. About the same timing mentioned here has a meaning similar to that of the above first embodiment.

As shown in FIG. 14 and FIG. 15, the branch portion 56 (the maxillary sinus) has a substantially triangular hollow shape (bag shape). There are individual differences in the shape of the maxillary sinus. In the present description, in the branch portion 56 (the maxillary sinus), a head top (superior) area 62, a jaw side (inferior) area 63, an anterior area 64 and a posterior area 65 are defined, whereby the description will be made. FIG. 15 shows respective areas in the branch portion 56 (the maxillary sinus) from a nasal cavity (hole 12) side in a perspective manner.

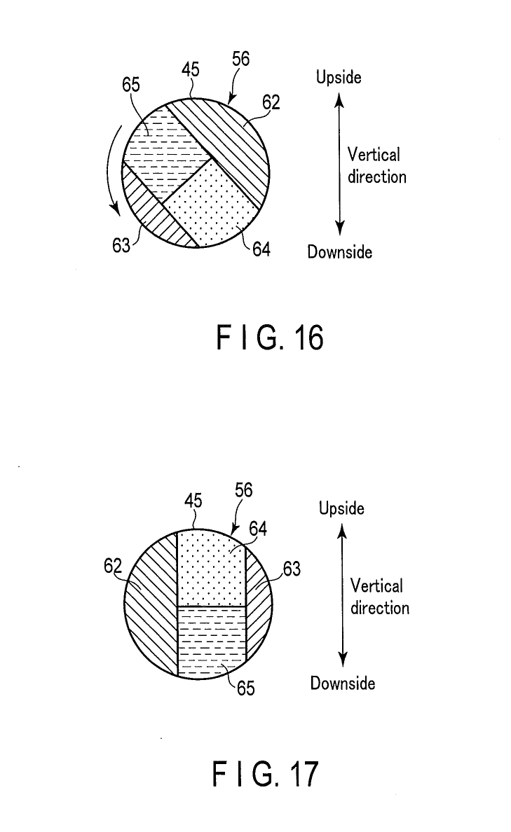

Prior to the rotation of the image 45 by the controller 14, as shown in FIG. 16, the image 45 obtained from the endoscope 16 shows the head top area 62 of the branch portion 56 on an upper right side of the image 45, shows the jaw side area 63 of the branch portion 56 on a lower left side, shows the posterior area 65 of the branch portion 56 on an upper left side, and shows the anterior area 64 of the branch portion 56 on a lower right side. In this state, the user sees the medical examinee who is in the recumbent posture in front of the user, and hence the user intuitively recognizes that the upside of the image 45 (a viewing field) is the anterior area 64 of the branch portion 56 (the maxillary sinus). However, in actual, the anterior area 64 of the branch portion 56 (the maxillary sinus) is present on a lower right side in the image 45, and hence the image does not match user's sense.

On the other hand, after the image 45 is rotated in the counterclockwise direction shown by an arrow in FIG. 16 by the controller 14, as shown in FIG. 17, the image shows the anterior area 64 of the branch portion 56 on the upside of the image 45, and shows the posterior area 65 of the branch portion 56 on the downside of the image 45. Furthermore, the jaw side area 63 of the branch portion 56 is shown on the right side of the image 45 and the head top area 62 of the branch portion 56 is shown on the left side of the image 45. Consequently, the upside of the image 45 recognized by the user can be matched with the upside of the vertical direction and the downside of the image 45 recognized by the user can be matched with the downside of the vertical direction. Therefore, the image 45 can be matched with the user's sense.

The user projects the endoscope insertion section 21 (the endoscope 16) from the distal portion 36 of the guide pipe 18, and inserts the endoscope insertion section 21 into the opening 56A of the branch portion 56 (the maxillary sinus) as shown in FIG. 14, while confirming the image 45 obtained from the endoscope 16. Thus, the user can observe the situation of the inside of the branch portion 56 (the maxillary sinus). After ending the observation of the inside of the branch portion 56, the user receives the endoscope insertion section 21 in the guide pipe 18, and twists the holding section as much as about 60° to 90° to direct the distal portion 36 of the guide pipe 18 to the jaw side. The controller 14 cancels the above-mentioned state where the image 45 is rotated, and returns the image to its original state. In this state, the user can safely remove the guide pipe 18 from the hole 12 (the nasal cavity) of the medical examinee.

### Second Example

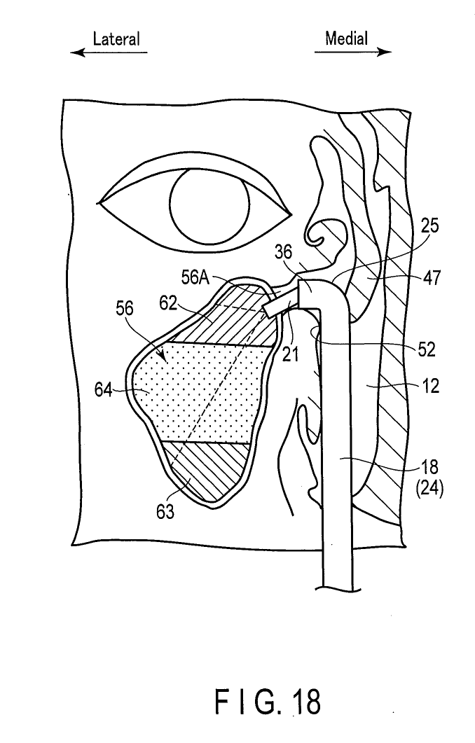

Next, there will be described the second example of the method for observing the branch portion of the hole which uses the endoscope system 11 of the present embodiment (the method for operating the endoscope system), with reference to FIG. 18 to FIG. 21. As shown in FIG. 18, a user inserts the guide pipe 18 into a hole 12 from right external nostril of a medical examinee (a subject) to observe the inside of the hole in diagnosis. It is assumed that the medical examinee in a recumbent posture (in a state of lying on a bed) has right maxillary sinus observed by the user who is a surgeon. Furthermore, the observation is performed in a recumbent posture second mode which is different from the above-mentioned second mode. A mode switching method is similar to that of the first example. Description of a part common with the first example is omitted.

The user performs a procedure similar to the first example so that the distal portion 36 of the guide pipe 18 can be opposed to (face) an opening 56A of a branch portion 56. In a state where the guide pipe 18 is inserted in a space 53 between a wall area 52 defining the periphery of the hole 12 and an obstacle 47 (such a state as shown in FIG. 7), the distal portion 36 of the guide pipe 18 (the distal constituting portion 26) is directed to a jaw side. At this time, similarly to the first example, the controller 14 acquires information on an extending direction (angle) of the distal portion 36 (the distal constituting portion 26) via the sensor 35 to store the information, whereby a jaw side (inferior) direction (angle) can be stored.

When bringing the distal portion 36 of the guide pipe 18 closer to the opening 56A of the branch portion 56, the user twists the holding section 17 as much as about 60° to 90° around the axial direction L similarly to the first example shown in FIG. 9 (strictly, in a reverse direction), while pushing the obstacle 47 aside toward a head top side with the elbow portion 25, whereby the guide pipe 18 can be rotated around the axial direction L to direct the distal portion 36 laterally from the jaw side. Thus, the distal portion 36 is laterally directed to push a second obstacle 48 aside and can be opposed to (face) the opening 56A of the branch portion 56. Consequently, the distal portion 36 can hold the second obstacle 48 in a state where the endoscope insertion section 21 (the endoscope 16) is insertable in the branch portion 56.

It is to be noted that the sensor 35 detects a degree of tilt of the vicinity of the distal end of the endoscope 16 to the jaw side, to always send the information to the controller. Consequently, also in circumstances where the distal portion 36 is laterally directed, the sensor 35 sends, to the controller 14, angular information indicating the degree of the tilt of the vicinity of the distal end of the endoscope 16 to the jaw side. The controller 14 judges whether the tilt of the guide pipe 18 (the distal portion 36) to the jaw side is a threshold value (e.g., 45°) or less, or is in excess of the threshold value (e.g., 45°).

The controller 14 rotates an image 45 displayed in the display 15 when obtaining the judgment result which is in excess of the above-mentioned threshold value. The controller 14 rotates the image 45 around its central area to match the downside of the vertical direction detected by the sensor 35 with the downside of the image 45 recognized by the user and to match the upside of the vertical direction detected by the sensor 35 with the upside of the image 45 recognized by the user. For example, as shown in FIG. 19, the image 45 is rotated as much as 180−θ° in a clockwise direction at this time, in which θ° is an angle to be formed by the direction L1 which comes closer to the elbow portion 25 of the guide pipe 18 and the downside of the vertical direction. On the other hand, when the controller 14 obtains the judgment result which is not more than the above-mentioned threshold value, the rotation of the image 45 displayed in the display 15 is not performed.

At this time, a timing to rotate the image 45 is about the same as a timing to rotate the guide pipe 18 around the axial direction L. About the same timing mentioned here has a meaning similar to that of the above first embodiment.

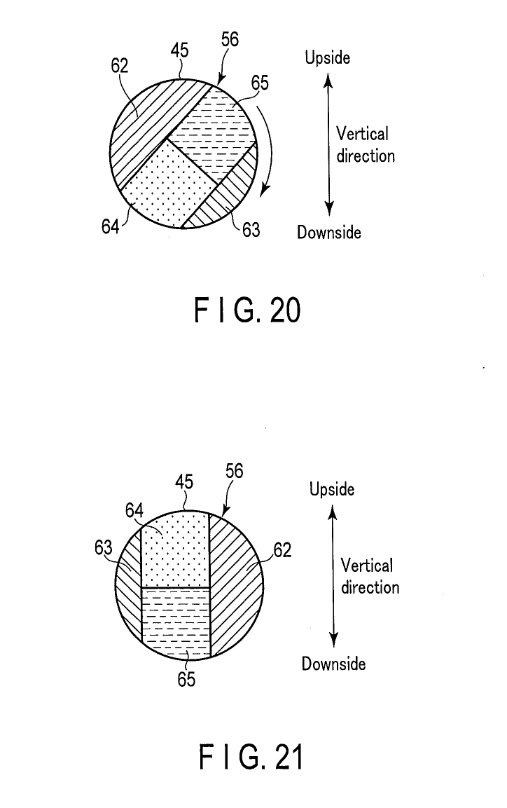

FIG. 19 shows respective areas in the branch portion 56 (the maxillary sinus) from a nasal cavity (hole 12) side in a perspective manner. Prior to the rotation of the image 45 by the controller 14, as shown in FIG. 20, the image 45 obtained from the endoscope 16 shows a head top area 62 of the branch portion 56 on an upper left side of the image 45, shows a jaw side area 63 of the branch portion 56 on a lower right side, shows a posterior area 65 of the branch portion 56 on an upper right side, and shows an anterior area 64 of the branch portion 56 on a lower left side. In this state, the user sees the medical examinee who is in the recumbent posture in front of the user, and hence the user intuitively recognizes that the upside of the image 45 (a viewing field) is the anterior area 64 of the branch portion 56 (the maxillary sinus). However, in actual, the anterior area 64 of the branch portion 56 (the maxillary sinus) is present on a lower left side in the image 45, and hence the image does not match user's sense.

On the other hand, after the image 45 is rotated in the clockwise direction shown by an arrow in FIG. 20 by the controller 14, as shown in FIG. 21, the image shows the anterior area 64 of the branch portion 56 on the upside of the image 45, and shows the posterior area 65 of the branch portion 56 on the downside of the image 45. Furthermore, the jaw side area 63 of the branch portion 56 is shown on the left side of the image 45 and the head top area 62 of the branch portion 56 is shown on the right side of the image 45. Consequently, the upside of the image 45 recognized by the user can be matched with the upside of the vertical direction and the downside of the image 45 recognized by the user can be matched with the downside of the vertical direction. Therefore, the image 45 can be matched with the user's sense.

The user projects the endoscope insertion section 21 (the endoscope 16) from the distal portion 36 of the guide pipe 18, and inserts the endoscope insertion section 21 into the opening 56A of the branch portion 56 (the maxillary sinus) as shown in FIG. 18, while confirming the image 45 obtained from the endoscope 16. Thus, the user can observe the situation of the inside of the branch portion 56 (the maxillary sinus). After ending the observation of the inside of the branch portion 56, the user receives the endoscope insertion section 21 in the guide pipe 18, and twists the holding section as much as about 60° to 90° to direct the distal portion 36 of the guide pipe 18 to the jaw side. The controller 14 cancels the above-mentioned state where the image 45 is rotated, and returns the image to its original state. In this state, the user can safely remove the guide pipe 18 from the hole 12 (the nasal cavity) of the medical examinee.

### Third Example

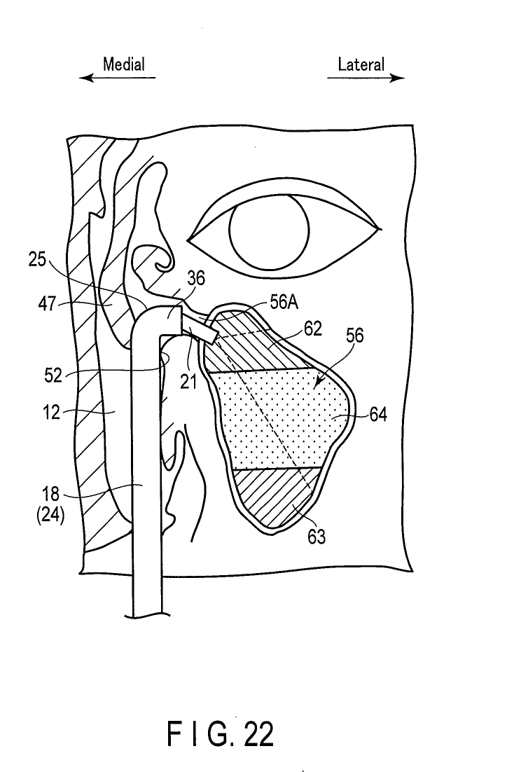

Next, there will be described the third example of the method for observing the branch portion of the hole which uses the endoscope system 11 of the present embodiment (the method for operating the endoscope system), with reference to FIG. 22 to FIG. 25. As shown in FIG. 22, a user inserts the guide pipe 18 into a hole 12 from left external nostril of a medical examinee (a subject) to observe the inside of the hole in diagnosis. It is assumed that the medical examinee in a seated posture (in a sitting state) has left maxillary sinus observed by the user who is a surgeon. Consequently, the observation is performed in a seated posture first mode which is different from the above-mentioned recumbent posture first mode of the first example. A mode switching method is similar to that of the first example. Description of a part common with the first example is omitted.

The user performs a procedure similar to the first example so that the distal portion of the guide pipe 18 can be opposed to (face) an opening 56A of a branch portion 56. In a state where the guide pipe 18 is inserted in a space 53 between a wall area 52 defining the periphery of the hole 12 and an obstacle 47 (such a state as shown in FIG. 7), the distal portion 36 of the guide pipe 18 (the distal constituting portion 26) is directed to the downside of a vertical direction.

In the same manner as in the first embodiment shown in FIG. 9, the user twists the holding section 17 as much as about 60° to 90° around the axial direction L, whereby the guide pipe 18 can be rotated around the axial direction L to direct the distal portion 36 laterally from the downside of the vertical direction. Thus, the distal portion 36 is laterally directed to push a second obstacle 48 aside and can be opposed to (face) the opening 56A of the branch portion 56. Consequently, the distal portion 36 can hold the second obstacle 48 in a state where the endoscope insertion section 21 (the endoscope 16) is insertable in the branch portion 56.

It is to be noted that the sensor 35 detects a degree of tilt of the vicinity of the distal end of the endoscope 16 to the downside of the vertical direction, to always send the information to the controller 14. Thus, also in circumstances where the distal portion 36 is laterally directed, the sensor 35 sends, to the controller 14, angular information indicating the degree of the tilt of the vicinity of the distal end of the endoscope 16 to the downside of the vertical direction. The controller 14 judges whether the tilt of the guide pipe 18 (the distal portion 36) to the downside of the vertical direction is a threshold value (e.g., 45°) or less, or is in excess of the threshold value (e.g., 45°).

When the controller 14 obtains the judgment result which is in excess of the above-mentioned threshold value, the controller rotates an image 45 displayed in the display 15. The controller 14 rotates the image 45 around its central area to match the downside of the vertical direction detected by the sensor 35 with the downside of the image 45 recognized by the user and to match the upside of the vertical direction detected by the sensor 35 with the upside of the image 45 recognized by the user. For example, as shown in FIG. 23, the image 45 is rotated as much as θ′° in a counterclockwise direction at this time, in which θ′° is an angle to be formed by the direction L1 which comes closer to the elbow portion 25 of the guide pipe 18 and the upside of the vertical direction. On the other hand, when the controller 14 obtains the judgment result which is not more than the above-mentioned threshold value, the rotation of the image 45 displayed in the display 15 is not performed. At this time, a timing to rotate the image 45 is about the same as a timing to rotate the guide pipe 18 around the axial direction L. About the same timing mentioned here has a meaning similar to that of the above embodiment.

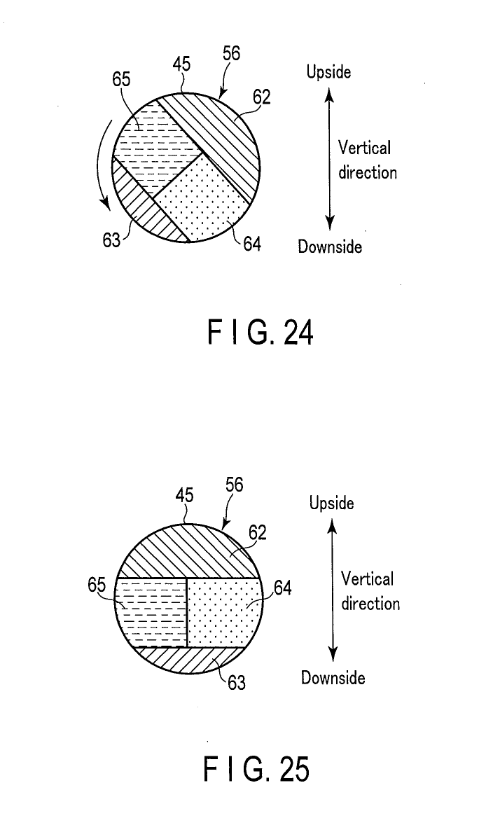

FIG. 23 shows respective areas in the branch portion 56 (the maxillary sinus) from the side of the nasal cavity in a perspective manner. Prior to the rotation of the image 45 by the controller 14, as shown in FIG. 24, the image 45 obtained from the endoscope 16 shows a head top area 62 of the branch portion 56 on an upper right side of the image 45, shows a jaw side area 63 of the branch portion 56 on a lower left side, shows a posterior area 65 of the branch portion 56 on an upper left side, and shows an anterior area 64 of the branch portion 56 on a lower right side. In this state, the user sees the medical examinee who is in the seated posture in front of the user, and hence the user intuitively recognizes that the upside of the image 45 (a viewing field) is the head top area 62 of the branch portion 56 (the maxillary sinus). However, in actual, the head top area 62 of the branch portion 56 (the maxillary sinus) is present on an upper right side in the image 45, and hence the image does not match user's sense.

On the other hand, after the image 45 is rotated in the counterclockwise direction shown by an arrow in FIG. 24 by the controller 14, as shown in FIG. 25, the image 45 shows the head top area 62 of the branch portion 56 on the upside of the image, and shows the jaw side area 63 of the branch portion 56 on the downside of the image 45. Furthermore, the anterior area 64 of the branch portion 56 is shown on the right side of the image 45 and the posterior area 65 of the branch portion 56 is shown on the left side of the image 45. Thus, the upside of the image 45 recognized by the user can be matched with the upside of the vertical direction and the downside of the image 45 recognized by the user can be matched with the downside of the vertical direction. Consequently, the image 45 can be matched with the user's sense.

Thus, the user can observe the situation of the inside of the branch portion 56 (the maxillary sinus). After ending the observation of the inside of the branch portion 56, the user receives the endoscope insertion section 21 in the guide pipe 18. The user twists the holding section 17 as much as about 60° to 90° to direct the distal portion 36 of the guide pipe 18 to the downside of the vertical direction (or the upside of the vertical direction). The controller 14 cancels the above-mentioned state where the image 45 is rotated, and returns the image to its original state. In this state, the user can safely remove the guide pipe 18 from the hole 12 (the nasal cavity) of the medical examinee.

### Fourth Example

Next, there will be described the fourth example of the method for observing the branch portion of the hole which uses the endoscope system 11 of the present embodiment (the method for operating the endoscope system). Similarly to the above third example, a user who is a surgeon can observe right maxillary sinus of a medical examinee in a seated posture (a sitting state). Furthermore, this observation is performed in a seated posture second mode which is different from the above-mentioned recumbent posture second mode of the second example. A mode switching method is similar to that of the first example. Description of a part common with the third example is omitted.

The user performs a procedure similar to the second example so that the distal portion 36 of the guide pipe 18 can be opposed to (face) an opening 56A of a branch portion 56. In a state where the guide pipe 18 is inserted in a space 53 between a wall area 52 defining the periphery of a hole 12 and an obstacle 47 (such a state as shown in FIG. 7), the distal portion 36 of the guide pipe 18 (the distal constituting portion 26) is directed to the downside of a vertical direction.

In the same manner as in the first embodiment shown in FIG. 9, the user twists the holding section 17 as much as about 60° to 90° around an axial direction L, whereby the guide pipe 18 can be rotated around the axial direction L to direct the distal portion 36 laterally from the downside of the vertical direction. Thus, the distal portion 36 is laterally directed to push a second obstacle 48 aside and can be opposed to (face) the opening 56A of the branch portion 56. Consequently, the distal portion 36 can hold the second obstacle 48 in a state where the endoscope insertion section 21 (the endoscope 16) is insertable in the branch portion 56.

It is to be noted that the sensor 35 detects a degree of tilt of the vicinity of the distal end of the endoscope 16 to the downside of the vertical direction, to always send the information to the controller 14. Thus, also in circumstances where the distal portion 36 is laterally directed, the sensor 35 sends, to the controller 14, angular information indicating the degree of the tilt of the vicinity of the distal end of the endoscope 16 to the downside of the vertical direction. The controller 14 judges whether the tilt of the guide pipe 18 (the distal portion 36) to the downside of the vertical direction is a threshold value (e.g., 45°) or less, or is in excess of the threshold value (e.g., 45°).

When the controller 14 obtains the judgment result which is in excess of the above-mentioned threshold value, the controller rotates an image 45 displayed in the display 15. The controller 14 rotates the image 45 around its central area to match the downside of the vertical direction detected by the sensor 35 with the downside of the image 45 recognized by the user and to match the upside of the vertical direction detected by the sensor 35 with the upside of the image 45 recognized by the user. For example, as shown in FIG. 23, the image 45 is rotated as much as θ′° in a clockwise direction at this time, in which θ′° is an angle to be formed by the direction L1 which comes closer to the elbow portion 25 of the guide pipe 18 and the upside of the vertical direction. On the other hand, when the controller 14 obtains the judgment result which is not more than the above-mentioned threshold value, the rotation of the image 45 displayed in the display 15 is not performed. At this time, a timing to rotate the image 45 is about the same as a timing to rotate the guide pipe 18. About the same timing mentioned here has a meaning similar to that of the above embodiment.

In a state prior to the rotation, in actual, a head top side of the branch portion 56 (the maxillary sinus) is present in an upper left side in the image 45, and hence the image does not match user's sense. After the image 45 is rotated in the clockwise direction by the controller 14, the image 45 shows a head top area 62 of the branch portion 56 on the upside of the image 45, and shows a jaw side area 63 of the branch portion 56 on the downside of the image 45. Furthermore, an anterior area 64 of the branch portion 56 is shown on the left side of the image 45 and a posterior area 65 of the branch portion 56 is shown on the right side of the image 45. Consequently, the image 45 can be matched with the user's sense.

Thus, the user can observe the situation of the inside of the branch portion 56 (the maxillary sinus). After ending the observation of the inside of the branch portion 56, the user receives the endoscope insertion section 21 in the guide pipe 18. The user twists the holding section 17 as much as about 60° to 90° to direct the distal portion 36 of the guide pipe 18 to the downside of the vertical direction (or the upside of the vertical direction). The controller 14 cancels the above-mentioned state where the image 45 is rotated, and returns the image to its original state. In this state, the user can safely remove the guide pipe 18 from the hole 12 (the nasal cavity) of the medical examinee.

According to the embodiment, conclusions can be made as follows. That is, a method for observing a branch portion of a hole uses an endoscope system 11 including a guide member having an elbow portion 25 and a distal portion 36 extending laterally from the elbow portion 25, an endoscope 16 whose orientation is adjustable by the guide member, a controller 14 which processes a signal acquired from the endoscope 16 to generate an image 45, and a display 15 which displays the image 45 generated by the controller 14. The method for observing the branch portion of the hole includes a step of pushing an obstacle 47 in a hole 12 aside with the elbow portion 25, a step of rotating the guide member around an axial direction L so that the distal portion 36 faces a branch portion 46 extending laterally to an extending direction of the hole 12, a step of rotating the image 45 to match an up-and-down direction of the image 45 with an up-and-down of a vertical direction, at about the same timing as a timing to rotate the guide member around the axial direction L, and a step of projecting the endoscope 16 from the distal portion 36 and inserting into the branch portion 46 while confirming the image 45.

According to this constitution, the obstacle 47 in the hole 12 can be pushed aside with the elbow portion 25, and even in circumstances where the obstacle 47 is present in the hole 12, the inside of the hole 12 and the branch portion 46 can be observed. Therefore, the observation can efficiently be performed. Furthermore, after the guide member is rotated, the up-and-down direction of the image 45 can be matched with the up-and-down of the vertical direction. Therefore, it is easy for the user to intuitively recognize the situation of the inside of the hole 12, and convenience for the user can improve. The user can be prevented from losing a sense of direction to the utmost, whereby it is possible to shorten observation time. Furthermore, it is possible to decrease the risk that the endoscope 16 is wrongly projected in an unintended direction, and it is therefore possible to prevent the endoscope 16 from hitting the wall area 52 of the hole 12 and damaging the wall area 52 of the hole 12.

The guide member includes a main body portion 24 that is continuous with the elbow portion 25, and the endoscope 16 can be disposed in a direction that forms a right angle or an obtuse angle to a direction which comes closer to the elbow portion 25 in the axial direction L of the main body portion 24. According to this constitution, the endoscope 16 can obtain a viewing field on a back side in accordance with a shape of the branch portion 46 of the hole 12.

A line segment A connecting the distal portion 36 and the elbow portion 25 forms a right angle or an obtuse angle to the direction which comes closer to the elbow portion 25 in the axial direction L of the main body portion 24. According to this constitution, when the obstacle 47 is pushed aside with the elbow portion 25, the endoscope 16 can securely be protected by the elbow portion 25 so that the obstacle 47 does not interfere with the endoscope 16.

The endoscope system 11 includes a sensor 35 which is configured to detect an angle to be formed by the line segment A connecting the distal portion 36 and the elbow portion 25 to the vertical direction. In the step of rotating the image 45, the controller 14 rotates the image 45 to match the up-and-down direction of the image 45 with the up-and-down of the vertical direction when angular information obtained from the sensor 35 is in excess of a predetermined threshold value. According to this constitution, the controller 14 automatically rotates the image 45 in a direction that matches user's intuition. Therefore, the convenience for the user can improve, and the observation time can shorten.

In the step of rotating the guide member around the axial direction L, the distal portion 36 of the guide member rides over a second obstacle 48 provided before the branch portion 46 in the hole 12, to obtain a state where the endoscope 16 is insertable in the branch portion 46. According to this constitution, the second obstacle 48 can be removed by the step of rotating the guide member around the axial direction. Consequently, even in circumstances where the second obstacle 48 which is different from the obstacle 47 is present, the observation of the branch portion 46 can smoothly be performed, and the observation can efficiently be performed.

In the step of pushing the obstacle 47 in the hole 12 aside with the elbow portion 25, the elbow portion 25 and the distal portion 36 are inserted into a space between the wall area 52 defining the periphery of the hole 12 and the obstacle 47 so that the line segment A connecting the distal portion 36 and the elbow portion 25 is substantially parallel with a plane D along which the obstacle 47 extends. According to this constitution, in the step of pushing the obstacle 47 in the hole 12 aside with the elbow portion 25, the distal portion 36 does not interfere with the obstacle 47 or the wall area 52, and an operation can smoothly be advanced.

The hole 12 is a nasal cavity, the branch portion 46 is a maxillary sinus extending laterally to an extending direction of the nasal cavity, the obstacle 47 is middle nasal concha, and the second obstacle 48 is an uncinate process. According to this constitution, when observation targets are the nasal cavity and maxillary sinus of a human body, the observation can smoothly be performed without damaging the middle nasal concha, the wall area defining the periphery of the nasal cavity, mucosa that is present around the observation target, or the like, in a step of pushing the middle nasal concha aside.

### Second Embodiment

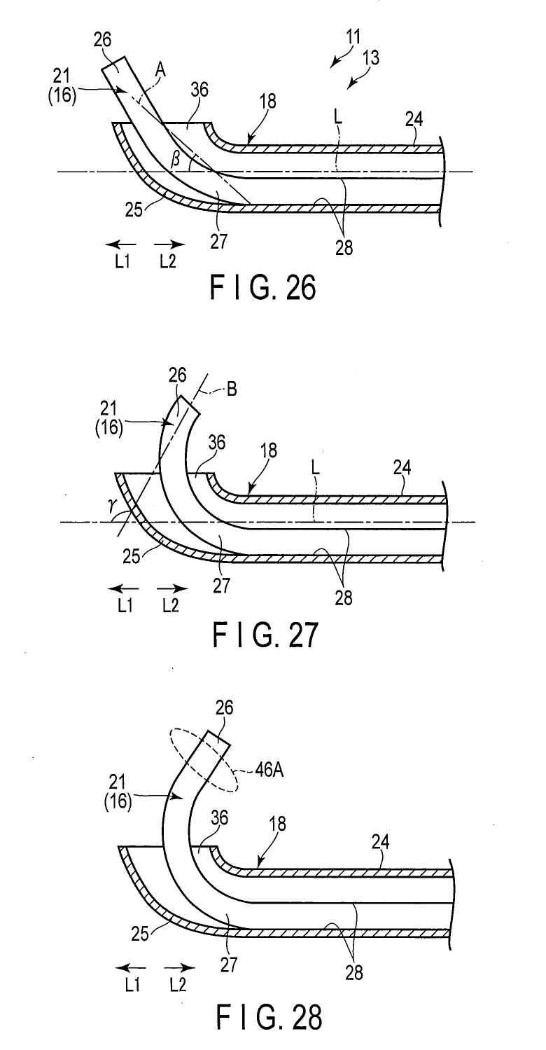

Next, there will be described an endoscope system 11 of a second embodiment and a method for observing a branch portion of a hole which uses the endoscope system, with reference to FIG. 26 to FIG. 29. Here, a part different from the first embodiment is mainly described, and drawing and description of a part common with the first embodiment are omitted.

As shown in FIG. 26, the guide pipe 18 substantially forms a “J”-shape as a whole, and forms a cylindrical shape halfway bent in the form of an elbow. The guide pipe 18 includes a main body portion 24 having one end portion attached to a holding section 17, an elbow portion 25 provided in the other end portion of the main body portion 24, and a distal portion 36 protruding from the elbow portion 25 in a direction away from the main body portion 24. In the present embodiment, an inner diameter of the distal portion 36 is larger than an inner diameter of the main body portion 24. That is, a diameter of the guide pipe 18 enlarges from the main body portion 24 toward the distal portion 36. An angle β to be formed by a line segment A connecting the distal portion 36 and the elbow portion 25 to a direction L1 which comes closer to the elbow portion 25 in an axial direction L (a central axis direction) of the main body portion 24 is, for example, an acute angle, more specifically 0°<β<90°, and preferably 0°<β<45°. The guide pipe 18 is one example of a guide member.

In the present embodiment, the inner diameter of the distal portion 36 increases, and hence an opening portion in the distal portion 36 is larger than that of the first embodiment. Thus, a configuration of an endoscope insertion section 21 (an endoscope 16) is similar to that of the first embodiment, but as shown in FIG. 27, the endoscope insertion section 21 can be bent, i.e., bended at an angle larger than that of the first embodiment, on the basis of an operation of a curve operation section 23. In a state where the endoscope insertion section 21 is bended, an angle γ to be formed by a central axis B of a distal constituting portion 26 and the axial direction L of the main body portion 24 of the guide pipe 18 is, for example, a right angle or an obtuse angle, and is more specifically 90°≤γ≤120°. Therefore, in the present embodiment, the endoscope insertion section 21 (the endoscope 16) can be disposed (can be bent) in a direction that forms the right angle or the obtuse angle to the direction L1 which comes closer to the elbow portion 25 on the basis of a shape of the distal portion 36 formed in the inner diameter larger than the inner diameter of the main body portion 24. Consequently, when a branch portion 46 of a hole 12 is observed, the branch portion 46 can be observed on the same conditions as in the first embodiment. An angle of an image 45 to be rotated clockwise or counterclockwise is not restricted to such a preset angle as described above, and as in the first example to the fourth example of the first embodiment, each appropriate rotation angle may be calculated on the basis of an angle at which the endoscope insertion section 21 (the guide pipe 18 and the distal constituting portion 26) is presently disposed, to rotate the image 45.

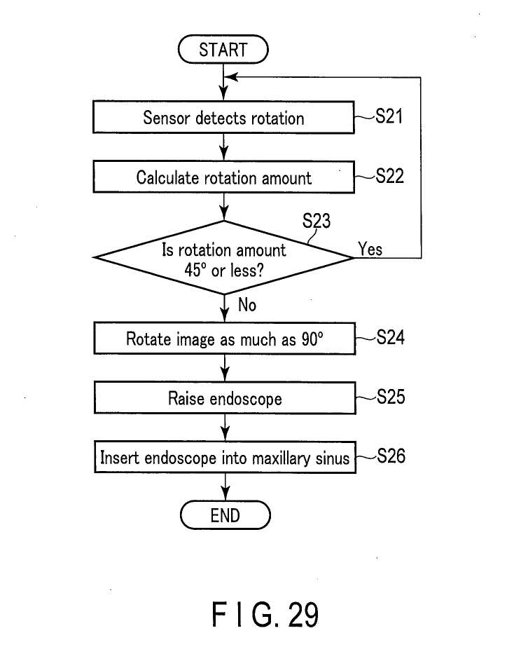

There will be described the method for observing the branch portion of the hole which uses the endoscope system 11 of the present embodiment. A flowchart of FIG. 29 shows respective steps of the present observing method, and the description will be made with reference to this flowchart. Here, there is described an example where the method for observing the branch portion of the hole is applied to observation of a paranasal sinus (a maxillary sinus) extending laterally from a nasal cavity of a medical examinee. It is assumed that the medical examinee who sits up on a seat plane receives the observation from a user who is a surgeon.

The user who is the surgeon can insert the guide pipe 18 into the hole 12 (a nasal cavity) from an external nostril of the medical examinee (a subject) in diagnosis. Similarly to the first embodiment, the user can insert the guide pipe 18 into a space 53 between a wall area 52 defining the periphery of the hole 12 and an obstacle 47. Thus, the distal portion 36 of the guide pipe 18 can be advanced toward a posterior side of a head of the medical examinee, while directing the distal portion 36 of the guide pipe 18 toward the downside of a vertical direction to push the obstacle aside with the elbow portion 25. At this time, similarly to the positional relation of the first embodiment shown in FIG. 8, the elbow portion 25 and the distal portion 36 have the relation that the line segment A connecting the distal portion 36 and the elbow portion 25 is substantially parallel with the plane D along which the obstacle 47 extends, also in the second embodiment.

The user may advance a distal end of the guide pipe 18 toward the head posterior side of the medical examinee, while directing the distal end of the guide pipe 18 toward the upside of the vertical direction to push the obstacle 47 aside with the distal portion 36. When advancing and retreating the guide pipe 18 in a state where the distal portion 36 of the guide pipe 18 is directed to the downside of the vertical direction (or the upside of the vertical direction) in this way, there is obtained a situation closely similar to a state where a person walks looking downward (or upward), and hence user's intuition is matched. That is, the upside of a viewing field (the image) corresponds to a head posterior side of the medical examinee (an advancing direction side), the downside of the viewing field (the image) corresponds to a head anterior side of the medical examinee (a side reverse to an advancing direction), the right and left correspond to those of the medical examinee as they are, and hence the closely similar situation is obtainable.

When bringing the distal portion 36 of the guide pipe 18 closer to an opening 46A of the branch portion 46, the user twists the holding section 17 as much as about 60° to 90° around the axial direction L as shown in FIG. 9, while pushing the obstacle 47 aside with the elbow portion 25, whereby the guide pipe 18 can be rotated around the axial direction L to direct the distal portion 36 laterally from the downside of the vertical direction. Thus, the distal portion 36 is laterally directed to ride over a second obstacle 48 (an uncinate process) and can be opposed to (face) the opening 46A of the branch portion 46. When directing the distal portion of the guide pipe 18 toward the upside of the vertical direction to advance the distal portion, the holding section 17 is twisted as much as about 60° to 90° around the axial direction L, whereby the distal portion 36 can be opposed to the opening 46A of the branch portion 46 (the maxillary sinus) while riding over the second obstacle 48 with the laterally directed distal portion 36.

In circumstances where the distal portion 36 is laterally directed, a sensor 35 can detect that the guide pipe 18 (the distal constituting portion 26) is rotated (step S21). The sensor 35 then detects an angle at which the distal portion 36 of the guide pipe 18 is rotated (tilted) to the downside of the vertical direction, to send angular information of the guide pipe 18 to a controller 14. The controller 14 calculates a rotation amount (a rotation angle) of the guide pipe 18 (the distal constituting portion 26) from the downside of the vertical direction on the basis of the angular information from the sensor 35 (step S22). The controller 14 judges whether the above rotation amount of the guide pipe 18 is a threshold value (e.g., 45°) or less or is in excess of the threshold value (e.g., 45°) (step S23), and the controller rotates an image displayed in a display 15 as much as 90° in a case where the above rotation amount of the guide pipe 18 is in excess of threshold value (e.g., 45°) (step S24). At this time, in a direction in which the image 45 rotates, the upside of the image 45 corresponds to the upside of the vertical direction and the downside of the image 45 corresponds to the downside of the vertical direction. As shown in FIG. 12, in a situation prior to the rotation of the image 45, an up-and-down direction of the image 45 extends along an anterior posterior direction, and hence the situation is not similar to a state where the person turns in a lateral direction. Therefore, the image does not match the user's intuition. On the other hand, as shown in FIG. 13, there is obtained a situation closely similar to a state where the person turns in the lateral direction (to the right side), after the image 45 is rotated in the step S24. That is, the upside of the image corresponds to the upside of the vertical direction and the downside of the image corresponds to the downside of the vertical direction. Furthermore, the left side of the image 45 corresponds to the head posterior side of the medical examinee (the advancing direction side) and the right side of the image 45 corresponds to the head anterior side of the medical examinee. Consequently, the user can intuitively recognize the situation of the inside of the hole 12.

In a case where the above rotation amount of the guide pipe 18 is not more than the threshold value (e.g., 45°), the image 45 to be displayed in the display 15 is not changed, but the sensor 35 continues to monitor presence/absence of the rotation of the guide pipe 18 (the step S21). After the image 45 is rotated, the up-and-down direction of the image 45 are matched with the up-and-down of the vertical direction, and hence the user can intuitively recognize the situation of the inside of the hole 12. In this state, the user noticeably bends the endoscope insertion section 21 toward the back side to bend the endoscope insertion section 21 (step S25). At this time, the image 45 obtainable from the endoscope 16 matches the user's intuition, and hence even when bending the endoscope insertion section 21, the disadvantage that the user loses a sense of direction or the like is hard to occur. In this state, the user utilizes the image 45 to minutely adjust a tilt of the endoscope insertion section 21 or a position of the distal constituting portion 26 as required. As shown in FIG. 28, the user projects the endoscope insertion section 21 (the endoscope 16) from the distal portion 36 of the guide pipe 18. The operation of bending the endoscope insertion section 21 (the step S25) and the operation of projecting the endoscope insertion section 21 (the endoscope 16) from the distal portion 36 may alternately be performed by small degrees. As shown in FIG. 28, the user inserts the endoscope insertion section into the opening 46A of the branch portion 46 (the maxillary sinus) (step S26). Consequently, the user can observe the situation of the inside of the branch portion 46 of the medical examinee. After ending the observation of the inside of the branch portion 46, the user receives the endoscope insertion section 21 in the guide pipe 18 and twists the holding section 17 as much as about 60° to 90° to direct the distal portion 36 of the guide pipe 18 to the downside of the vertical direction (or the upside of the vertical direction). It is preferable that the controller 14 returns the image 45 to its state prior to the rotation to match the user's intuition after the distal portion 36 of the guide pipe 18 is directed to the downside of the vertical direction.

More specifically, the sensor 35 detects the presence/absence of the rotation of the guide pipe 18 (the distal constituting portion 26), and returns the image 45 to its state prior to the rotation in a case where the angle (tilt) of the rotated guide pipe 18 (distal constituting portion 26) is not more than the 45° from the downside of the vertical direction (or the upside of the vertical direction). Also in the state where the distal portion 36 of the guide pipe 18 is directed to the downside of the vertical direction (or the upside of the vertical direction), the image 45 obtainable from the endoscope 16 matches the user's intuition. Consequently, the user can safely remove the guide pipe 18 from the hole 12 (the nasal cavity).

According to the present embodiment, the line segment A connecting the distal portion 36 and the elbow portion 25 forms the acute angle to the direction L1 which comes closer to the elbow portion 25 in the axial direction L of the main body portion 24, and the endoscope 16 is bendable to form the right angle or the obtuse angle to the direction L1 which comes closer to the elbow portion 25 in the axial direction L of the main body portion 24. According to this constitution, in a state before bending the endoscope 16 toward the back side, the endoscope 16 is directed toward the front side. Consequently, when inserting the endoscope 16 into the hole 12 and advancing the endoscope in the hole 12, the user can advance the endoscope 16 while confirming the front side by use of the image 45 obtained from the endoscope. Therefore, convenience for the user can improve, and it is possible to shorten time required until the observation target is reached.

At this time, the method includes, after the step of rotating the image 45, the step of bending the endoscope 16 to form the right angle or the obtuse angle to the direction which comes closer to the elbow portion 25 in the axial direction L of the main body portion 24. According to this constitution, it is possible to bend the endoscope 16 in a state where the image 45 is rotated. When the endoscope 16 is bended, the endoscope can be prevented from coming in contact with the wall area 52 around the hole 12, or the like, and the observation can safely be performed. In the steps prior to this bending step, the endoscope 16 is not bended and the endoscope 16 does not laterally project outside. That is, in the state before bending the endoscope 16, a height of the guide member can be minimized, and guiding properties of the guide member in the hole 12 can suitably improve.

Hitherto, the embodiments and respective modifications have specifically be described with reference to the drawings, but this invention is not restricted to the above-mentioned embodiments, and constituent elements can be modified and embodied without departing from the gist of the invention. In the above embodiments, a scanning type endoscope is used, but needless to say, in the method for observing the branch portion of the hole and the method for operating the system, a non-scanning type endoscope (a so-called usual endoscope) which does not have the rotating unit 32 is also usable. Examples of the hole 12 and branch portion 46 of the medical examinee (the subject) include the nasal cavity and maxillary sinus, but needless to say, the above endoscope system is usable in observation of another hole of a human body, e.g., an urethra, an urinary bladder or the like. In the above embodiments, there have been described the examples of the observation of left nasal cavity and paranasal sinus, but needless to say, the present invention is also applicable to observation of right nasal cavity and maxillary sinus by reversing a rotating direction of the guide pipe 18 and a rotating direction of the image 45.

Furthermore, needless to say, the method for observing the branch portion of the hole is usable not only in inspection and observation of the human body but also in observation of the inside of a branch portion 46 of a hole 12 (a pipe or a duct) in a mechanical structure.

Further in the above respective embodiments, control to rotate the image 45 as much as 90° is automatically executed by the detection of the rotation angle by the sensor 35, but needless to say, the rotation of the image 45 may manually be performed by a switch operation or the like. In this case, for example, it is preferable to provide a switch (a button) in a case of the holding section 17 or the controller 14, and a rotating operation of the image 45 may suitably be performed by user's operation of the switch. Furthermore, in the case of manually performing the rotation of the image 45, the user may rotate the image 45 by performing the switch operation immediately before the rotation of the guide pipe 18 around the axial direction L, simultaneously with the rotation, or immediately after the rotation. In the above embodiments, the medical examinee sitting up on the seat plane receives the observation, but also in a case of performing observation with respect to a medical examinee in a lying state, intuitive observation can similarly be performed by rotating the image 45 to match the up-and-down direction of the image with the up-and-down of the vertical direction.

Furthermore, one endoscope system 11 is achievable by suitably combining constituent elements of the above-mentioned different embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method for observing a branch portion of a hole which uses an endoscope system comprising a guide member having an elbow portion and a distal portion extending laterally from the elbow portion, an endoscope whose orientation is adjustable by the guide member, a controller which processes a signal acquired from the endoscope to generate an image, and a display which displays the image generated by the controller,
the method for observing the branch portion of the hole comprising:
a step of pushing an obstacle in the hole aside with the elbow portion;
a step of rotating the guide member around an axial direction so that the distal portion faces the branch portion extending laterally to an extending direction of the hole;
a step of rotating the image to match an up-and-down direction of the image with an up-and-down of a vertical direction, at about the same timing as a timing to rotate the guide member around the axial direction; and
a step of projecting the endoscope from the distal portion and inserting into the branch portion while confirming the image.

2. The method for observing the branch portion of the hole according to claim 1,
wherein the endoscope system comprises a sensor which is configured to detect an angle to be formed by a line segment connecting the distal portion and the elbow portion to the vertical direction, and
in the step of rotating the image, the controller rotates the image to match the up-and-down direction of the image with the vertical direction when angular information obtained from the sensor is in excess of a predetermined threshold value.

3. The method for observing the branch portion of the hole according to claim 2,
wherein in the step of rotating the image, the controller rotates the image around its central area as much as a preset angle, to match the up-and-down direction of the image with the vertical direction.

4. The method for observing the branch portion of the hole according to claim 2,
wherein in the step of rotating the image, the controller obtains an angle to rotate the image on the basis of angular information obtained from the sensor, and rotates the image around its central area as much as the angle, to match the up-and-down direction of the image with the vertical direction.

5. The method for observing the branch portion of the hole according to claim 2,
wherein the guide member includes a main body portion that is continuous with the elbow portion, and
the endoscope is to be disposed so that a distal constituting portion extends in a direction that forms a right angle or an obtuse angle to a direction which comes closer to the elbow portion in the axial direction of the main body portion.

6. The method for observing the branch portion of the hole according to claim 5,
wherein the line segment connecting the distal portion and the elbow portion forms a right angle or an obtuse angle to the direction which comes closer to the elbow portion in the axial direction of the main body portion.

7. The method for observing the branch portion of the hole according to claim 5,
wherein the line segment connecting the distal portion and the elbow portion forms an acute angle to the direction which comes closer to the elbow portion in the axial direction of the main body portion, and
the endoscope is bendable so that an extending direction of the distal constituting portion forms a right angle or an obtuse angle to the direction which comes closer to the elbow portion in the axial direction of the main body portion.

8. The method for observing the branch portion of the hole according to claim 5, comprising, after the step of rotating the image, a step of bending the endoscope so that an extending direction of the distal constituting portion forms a right angle or an obtuse angle to the direction which comes closer to the elbow portion in the axial direction of the main body portion.

9. The method for observing the branch portion of the hole according to claim 1,
wherein in the step of rotating the guide member around the axial direction, the distal portion of the guide pipe engages with a second obstacle provided before the branch portion in the hole to ride over the second obstacle, and the second obstacle is held in a state where the endoscope is insertable in the branch portion.

10. The method for observing the branch portion of the hole according to claim 1,
wherein in the step of pushing the obstacle in the hole aside with the elbow portion, the elbow portion and the distal portion are inserted into a space between a wall area defining the periphery of the hole and the obstacle so that a line segment connecting the distal portion and the elbow portion is substantially parallel with an extending direction of the obstacle.

11. The method for observing the branch portion of the hole according to claim 9,
wherein the hole is a nasal cavity,
the branch portion is a maxillary sinus extending laterally to an extending direction of the nasal cavity,
the obstacle is middle nasal concha, and
the second obstacle is an uncinate process.

12. A method for operating an endoscope system comprising a guide member having an elbow portion and a distal portion extending laterally from the elbow portion, an endoscope whose orientation is adjustable by the guide member, a controller which processes a signal acquired from the endoscope to generate an image, a display which displays the image generated by the controller, and a sensor which is configured to detect an angle to be formed by a line segment connecting the distal portion and the elbow portion to a vertical direction,
the method for operating the endoscope system comprising:
a step of rotating the guide member around an axial direction so that the distal portion faces a branch portion extending laterally to an extending direction of a hole;
a step in which the sensor detects the rotation of the guide member;
a step of judging whether or not a rotation amount of the guide member is in excess of a predetermined threshold value; and
a step of rotating the image to match an up-and-down direction of the image with the vertical direction in a case where the rotation amount of the guide member is in excess of the predetermined threshold value.
